(19) United States
(12) Patent Application Publication
(10) Pub. No.: US 2010/0010217 A1
(43) Pub. Date: Jan. 14, 2010

(54) METHODS FOR THE PREPARATION OF IMIDAZOLE-CONTAINING COMPOUNDS

(76) Inventors: Nicholas M. Valiante, Fremont, CA (US); James Sutton, Pleasanton, CA (US); Jiong Lan, Moraga, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/294,225

(22) PCT Filed: Mar. 23, 2007
(86) PCT No.: PCT/US07/64855

§ 371 Date: Mar. 6, 2009

Related U.S. Application Data
(60) Provisional application No. 60/785,661, filed on Mar. 23, 2006.

(51) Int. Cl. C07D 473/32; C07D 471/14; C07D 471/02; C07D 215/02; C07D 233/88

(52) U.S. Cl. 544/277; 546/82; 546/118; 546/155; 548/331.5

(57) ABSTRACT

The present invention generally relates to methods for the preparation of compounds that contain imidazole moieties. In some embodiments, the methods include the reaction of a diamine with a dichloroimmonium compound to produce the imidazole moiety. In some embodiments, the methods are employed to prepare compounds having the Formulas II, II or III below: I II III wherein the constituent variables are as described herein.

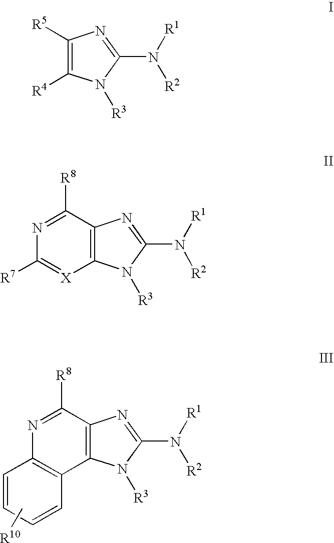

### CROSS REFERENCE TO RELATED APPLICATIONS

This application claims the benefit under 35 U.S.C. § 119(e) of U.S. Provisional Application Ser. No. 60/785,661, filed on Mar. 23, 2006, which is hereby incorporated by reference in its entirety.

### FIELD OF THE INVENTION

The present invention generally relates to methods for the preparation of compounds that contain imidazole moieties. In some embodiments, the methods include the reaction of a diamine with a dichloroimmonium compound to produce the imidazole moiety. In some embodiments, the methods are used to prepare compounds that are small molecule immune potentiators (SMIPs), that are capable of stimulating or modulating an immune response in a subject, and that can be used as immunotherapeutic agents for proliferative diseases, infectious diseases, autoimmune diseases, allergies, and/or asthma.

### BACKGROUND OF THE INVENTION

Issued U.S. Pat. Nos. 4,689,338, 5,389,640, 5,268,376, 4,929,624, 5,266,575, 5,352,784, 5,494,916, 5,482,936, 5,346,905, 5,395,937, 5,238,944, 5,525,612, and 6,110,929, and WO 99/29693 disclose imidazoquinoline compounds of the general structure (a) for use as “immune response modifiers”:

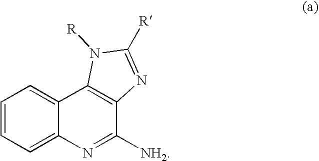

Each of these references is hereby incorporated by reference in its entirety and for all purposes as if fully set forth herein.

U.S. Pat. No. 6,083,505, describes specific imidazoquinolines for use as adjuvants. WO 03/097641 discloses the use of certain imidazoquinolines and salts thereof for the treatment of certain protein kinase dependent diseases and for the manufacture of pharmaceutical preparations for the treatment of diseases.

Immune response to certain antigens can be enhanced through the use of immune potentiators, known as vaccine adjuvants. Such adjuvants potentiate the immune response to specific antigens and are, therefore, the subject of considerable interest and study within the medical community.

Research has resulted in the development of vaccines possessing antigenic epitopes that were previously impossible to produce. For example, currently available vaccine candidates include synthetic peptides mimicking numerous bacterial and viral antigens. The immune response to these purified antigens can be enhanced by coadministration of an adjuvant. Unfortunately, conventional vaccine adjuvants possess a number of drawbacks that limit their overall use and effectiveness. Moreover, many of the adjuvants currently available have limited utility because they include components that are not metabolized by humans. Additionally, most adjuvants are difficult to prepare and may require time-consuming procedures and, in some cases, the use of elaborate and expensive equipment to formulate a vaccine and adjuvant system.

Immunological adjuvants are described in “Current Status of Immunological Adjuvants”, Ann. Rev. Immunol., 1986, 4, pp. 369-388, and “Recent Advances in Vaccine Adjuvants and Delivery Systems” by Derek T O'Hagan and Nicholas M. Valiante. See also U.S. Pat. Nos. 4,806,352; 5,026,543; and 5,026,546 for disclosures of various vaccine adjuvants appearing in the patent literature. Each of these references is hereby incorporated by reference in its entirety and for all purposes as if fully set forth herein.

Efforts have been made to identify new immune modulators for use as adjuvants for vaccines and immunotherapies that would overcome the drawbacks and deficiencies of conventional immune modulators. In particular, an adjuvant formulation that elicits potent cell-mediated and humoral immune responses to a wide range of antigens in humans and domestic animals, but lacking the side effects of conventional adjuvants and other immune modulators, would be highly desirable. This need could be met by small molecule immune potentiators (SMIPs) because the small molecule platform provides diverse compounds for the selective manipulation of the immune response, necessary for increasing the therapeutic index immune modulators.

Novel sole-acting agents with varied capacities for altering levels and/or profiles of cytokine production in human immune cells are needed. Compounds with structural disparities will often elicit a desired response through a different mechanism of action, or with greater specificity to a target, such as a dendritic cell, modulating potency and lowering side effects when administered to a patient.

The immunosuppressive effect of cytostatic substances has rendered them useful in the therapy of autoimmune diseases such as multiple sclerosis, psoriasis and certain rheumatic diseases. Unfortunately, their beneficial effect has to be weighed against serious side effects that necessitate dosages that are too low. Furthermore, interruption of the treatment may be required.

Agents and/or combinations of active substances that result in significantly improved cytostatic or cytotoxic effects compared to conventional cytostatics e.g., vincristin, methotrexate, cisplatin, etc., are needed. With such agents and combinations, chemotherapies may be offered that combine increasing efficiency with a large reduction of side effects and therapeutic doses. Such agents and combination therapies may thus increase the therapeutic efficiency of known cytostatic drugs. In some embodiments, the compounds of the invention are used in combination with compounds that provide significantly improved cytostatic or cytotoxic effect compared to conventional cytostatic agents when administered alone. Additionally, cell lines that are insensitive to conventional chemotherapeutic treatment may also be susceptible to chemotherapy using combinations of active substances.

Improved methods for preparing therapeutics that serve to augment natural host defenses against viral and bacterial infections, or against tumor induction and progression, with reduced cytotoxicity, are needed. The present invention provides such methods, and further provides other related advantages. The current invention provides method of preparing therapeutic and prophylactic agents for treatment of disease states characterized by other immune deficiencies, abnormalities, or infections including autoimmune diseases and viral and bacterial infections responsive to compounds with the capacity to modulate cytokines and/or TNF-α.

### BRIEF SUMMARY OF THE INVENTION

The present invention provides methods for the preparation of compounds that contain imidazole moieties. In some embodiments, the methods include the reaction of a diamine with a dichloroimmonium compound to produce the imidazole moiety. In some embodiments, the methods are used to prepare compounds that are small molecule immune potentiators (SMIPs), that are capable of stimulating or modulating an immune response in a subject, and that can be used as immunotherapeutic agents for proliferative diseases, infectious diseases, autoimmune diseases, allergies, and/or asthma.

In one aspect, the invention provides methods for synthesizing a compound of Formula I:

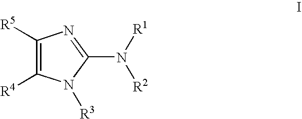

comprising:

reacting a compound of Formula IA:

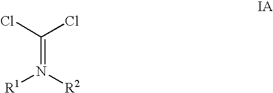

with a compound of Formula IB:

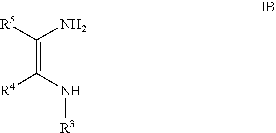

wherein:

R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;

or R¹ and R² are taken together to form a heterocyclyl or substituted heterocyclyl group;

R³ is selected from the group consisting of H, alkyl, substituted alkyl, hydroxy, alkoxy, substituted alkoxy, amino, substituted amino, acyl, and substituted carbonyl;

R⁴ and R⁵ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;

or R⁴ and R⁵ taken together form a heteroaryl, substituted heteroaryl, cycloalkyl, substituted cycloalkyl, heterocyclyl, or substituted heterocyclyl group.

In a further aspect, the invention provides methods of synthesizing a compound of Formula II:

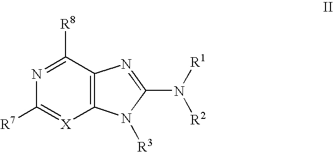

said method comprising the step of:

reacting a compound of Formula IA:

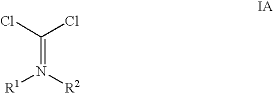

with a compound of Formula IIB:

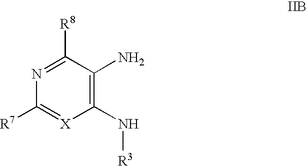

wherein,

X is N or CR⁶;

R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, hydroxy, alkoxy, substituted alkoxy, substituted alkyl, amino, substituted amino, acyl, and substituted carbonyl;

R⁶ and R⁷ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;

or R⁶ and R⁷ taken together form an aryl, substituted aryl, heteroaryl, substituted heteroaryl, cycloalkyl, substituted cycloalkyl, heterocyclyl, or substituted heterocyclyl group; and

R⁸ is selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some embodiments, wherein R⁸ is a —N(PMB)₂ group, the methods further include removing the PMB groups from R⁸ to form an amino group at R⁸. In some embodiments, wherein R⁸ is a halogen, the methods further include displacing the halogen with an amino or substituted amino group, to form a compound wherein R⁸ is an amino or substituted amino group. In some embodiments, wherein R⁸ is hydrogen, the methods further include reacting said compound of Formula II with an oxidizing agent, for example mCPBA or H₂O₂ to form an N→O (N-oxide) at the 5-position; and optionally then reacting the compound of Formula II having a N→O (N-oxide) at the 5-position, with a halogenating agent, to form a compound wherein R⁸ is a halogen.

In some embodiments, the methods further include synthesizing a compound of Formula IIB:

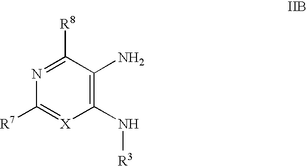

the synthesis comprising the steps of:

reacting a compound of Formula IIC:

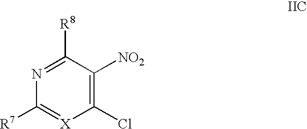

with a compound of formula H₂N—R³, to form a compound of Formula IID:

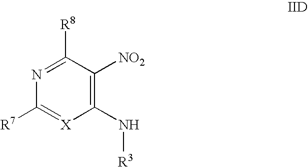

and reacting the compound of Formula IID with a hydrogenating agent. In some embodiments wherein R⁸ is a halogen, the methods further include synthesizing a compound of Formula IIC:

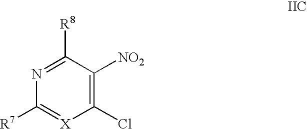

wherein R⁸ is chloro, said synthesis comprising the step of:

reacting a compound of Formula IIE:

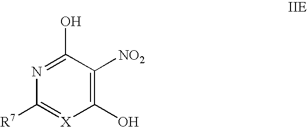

with a chlorinating agent. In some such embodiments, the methods further include synthesizing a compound of Formula IIE:

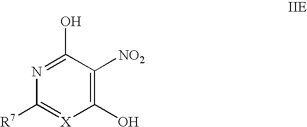

the synthesis comprising the step of:

reacting a compound of Formula IIF:

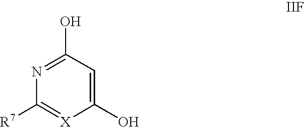

with a nitrosylating agent.

In a further aspect, the invention provides methods of synthesizing a compound of Formula III:

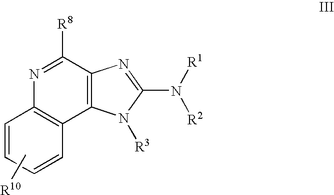

comprising:

reacting a compound of Formula IA:

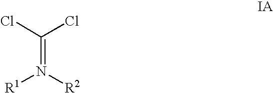

with a compound of Formula IIIB:

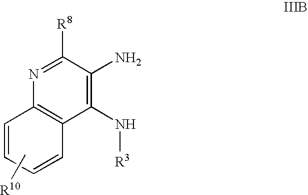

wherein:

R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, hydroxy, substituted alkyl, alkoxy, substituted alkoxy, amino, substituted amino, acyl, and substituted carbonyl;

R⁸ and R¹⁰ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some embodiments, wherein R⁸ is a halogen, the methods further include displacing the halogen with an amino or substituted amino group, to form a compound wherein R⁸ is an amino or substituted amino group. In some embodiments, wherein R⁸ is hydrogen, the methods further include reacting the compound of Formula III with an oxidizing agent, for example mCPBA or H₂O₂ to form an N→O (N-oxide) at the 5-position. In some such embodiments, the methods further include reacting the compound of Formula III with a halogenating agent, to form a compound wherein R⁸ is a halogen and X is N; and optionally displacing the halogen R⁸ with an amino or substituted amino group, to form a compound wherein R⁸ is an amino or substituted amino group.

In some embodiments, the methods further include synthesizing a compound of Formula IIIB:

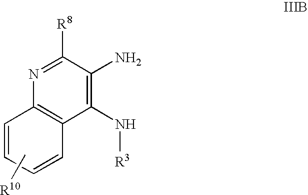

said synthesis comprising:

reacting a compound of Formula IIIC:

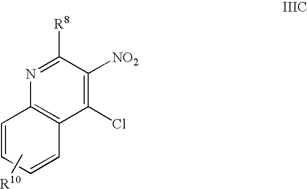

with a compound of formula H₂N—R³, to form a compound of Formula IIID:

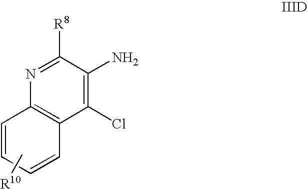

and reacting said compound of Formula IIID with a hydrogenating agent. In some embodiments, the methods further include synthesizing a compound of Formula IIIC:

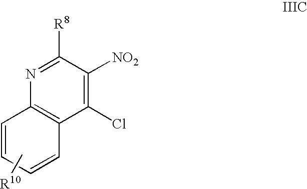

wherein R⁸ is chloro, said synthesis comprising the step of:

reacting a compound of Formula IIIE:

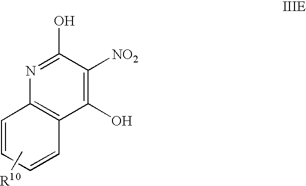

with a chlorinating agent. In some such embodiments, the methods further include synthesizing a compound of Formula IIIE:

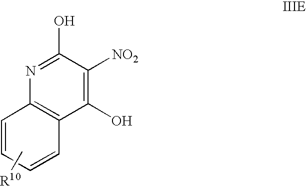

said synthesis comprising the step of:

reacting a compound of Formula IIIF:

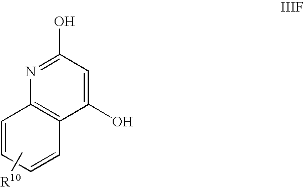

with a nitrosylating agent. In some such embodiments, the compound of Formula IA:

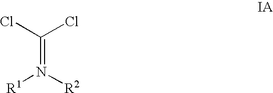

is prepared by reacting a compound of Formula IC:

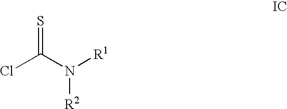

with phosgene or diphosgene.

In a further aspect, the invention provides methods for synthesizing a compound of Formula II:

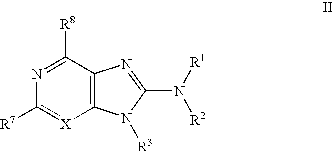

comprising:

reacting a compound of Formula ID:

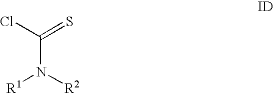

with a compound of Formula IIB:

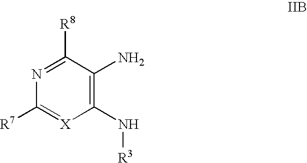

wherein,

X is N or CR⁶;

R¹ and R² are each independently selected from the group consisting of hydrogen, alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, hydroxy, alkoxy, substituted alkoxy, substituted alkyl, amino, substituted amino, acyl, and substituted carbonyl;

R⁶ and R⁷ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;

or R⁶ and R⁷ taken together form an aryl, substituted aryl, heteroaryl, substituted heteroaryl, cycloalkyl, substituted cycloalkyl, heterocyclyl, or substituted heterocyclyl group; and

R⁸ is selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some embodiments, R⁸ is a substituted amino group, preferably a-N(PMB)₂ group, and the methods further include removing the PMB groups from said R⁸ to form an amino group at R⁸. In some embodiments, wherein R⁸ is a halogen, the methods further include displacing said halogen with an amino or substituted amino group, to form a compound wherein R⁸ is an amino or substituted amino group. In some embodiments, wherein R⁸ is hydrogen, the methods further include reacting the compound of Formula II with an oxidizing agent, for example mCPBA or H₂O₂ to form an N→O (N-oxide) at the 5-position. In some such embodiments, the methods further include reacting the N-oxide with a halogenating agent, to form a compound wherein R⁸ is a halogen.

In some embodiments, the methods further include synthesizing a compound of Formula IIB:

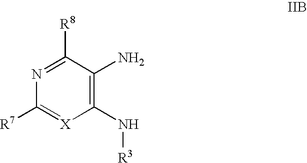

said synthesis comprising the step of:

reacting a compound of Formula IIC:

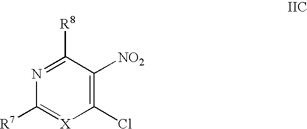

with a compound of formula H₂N—R³, to form a compound of Formula IID:

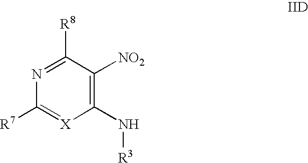

and reacting said compound of Formula IID with a hydrogenating agent.

In some embodiments, wherein R⁸ is a halogen, the methods further include reacting the compound of Formula IID with HN(PMB)₂, to form a compound wherein R⁸ is —N(PMB)₂.

In some embodiments, the methods further include synthesizing a compound of Formula IIC:

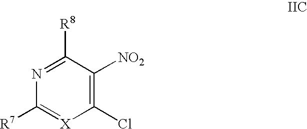

wherein R⁸ is chloro, the synthesis comprising the step of:

reacting a compound of Formula IIE:

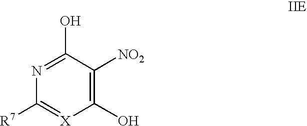

with a chlorinating agent.

In some embodiments, the methods further include synthesizing a compound of Formula IIE:

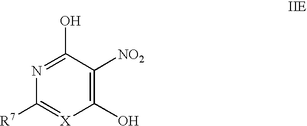

said synthesis comprising:

reacting a compound of Formula IIF:

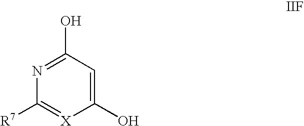

with a nitrosylating agent.

In a further aspect, the invention provides methods of synthesizing a compound of Formula III:

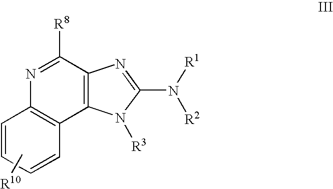

comprising:

reacting a compound of Formula ID:

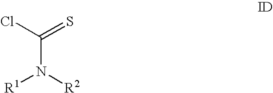

with a compound of Formula IIIB:

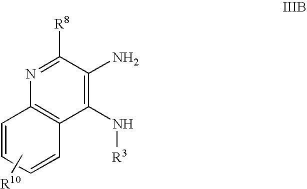

wherein:

R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, hydroxy, substituted alkyl, alkoxy, substituted alkoxy, amino, substituted amino, carbonyl, and substituted carbonyl;

R⁸ and R¹⁰ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some embodiments, wherein R⁸ is a —N(PMB)₂ group, the methods further include removing the PMB groups from R⁸ to form an amino group at R⁸. In some embodiments, wherein R⁸ is a —N₃ group, the methods converting the azide groups from R⁸ to form an amino group at R⁸.

In some embodiments, wherein R⁸ is a halogen, the methods further include displacing the halogen with an amino or substituted amino group, to form a compound wherein R⁸ is an amino or substituted amino group.

In some embodiments, wherein R⁸ is hydrogen, the methods further include reacting said compound of Formula III with an oxidizing agent, for example mCPBA or H₂O₂ to form an N→O (N-oxide) at the 5-position; and then optionally reacting the N-oxide with a halogenating agent, to form a compound wherein R⁸ is a halogen.

In some embodiments, the methods further include synthesizing a compound of Formula IIIB:

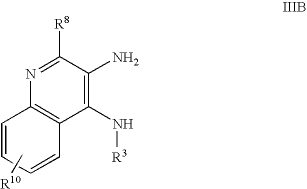

said synthesis comprising:

reacting a compound of Formula IIIC:

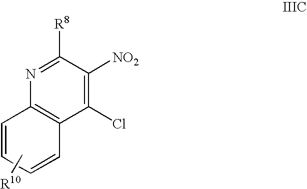

with a compound of formula H₂N—R³, to form a compound of Formula IIID:

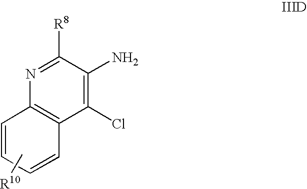

and reacting the compound of Formula IIID with a hydrogenating agent.

In some embodiments, the methods further include reacting the compound of Formula IIID with HN(PMB)₂, to form a compound wherein R⁸ is —N(PMB)₂.

In some embodiments, the methods further include synthesizing a compound of Formula IIIC:

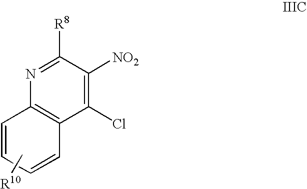

wherein R⁸ is chloro;
said synthesis comprising the step of:

reacting a compound of Formula IIIE:

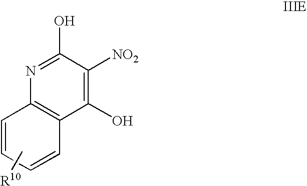

with a chlorinating agent.

In some embodiments, the methods further include synthesizing a compound of Formula IIIE:

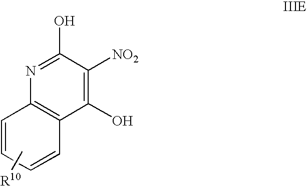

said synthesis comprising:

reacting a compound of Formula IIIF:

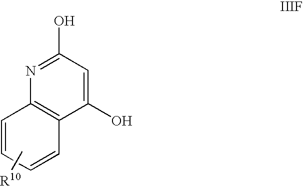

with a nitrosylating agent.

In some embodiments, the methods described herein further include the step of purifying a compound prepared by the methods described herein. In more particular embodiments, said purifying includes one or more of chromatography, distillation, recrystallization, filtration, extraction, and/or drying or azeotroping.

In a further aspect, the invention provides methods of inducing an immune response in a subject, comprising administering a compound, prepared according to the methods described herein, to the subject in an amount sufficient to induce an immune response in the subject. In some such embodiments, the immune response is TLR7 and/or TLR8 related.

### DETAILED DESCRIPTION OF THE INVENTION

The present invention provides methods for preparing compounds that contain imidazole moieties. In some embodiments, the compounds are small molecule immune potentiators (SMIPs), that are capable of stimulating or modulating an immune response in a subject, and that can be used as immunotherapeutic agents for proliferative diseases, infectious diseases, autoimmune diseases, allergies, and/or asthma.

In a first aspect, the invention provides methods of synthesizing a compound of Formula I:

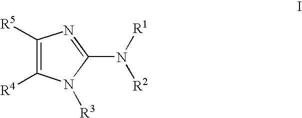

comprising:

reacting a compound of Formula IA:

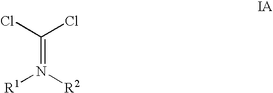

with a compound of Formula IB:

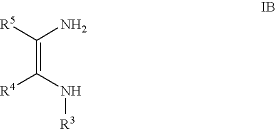

wherein:

R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, substituted alkyl, hydroxy, alkoxy, substituted alkoxy, amino, substituted amino, acyl, and substituted carbonyl;

R⁴ and R⁵ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;

or R⁴ and R⁵ taken together form a heteroaryl, substituted heteroaryl, cycloalkyl, substituted cycloalkyl, heterocyclyl, or substituted heterocyclyl group.

In some such embodiments, the compound of Formula IA further includes a negatively charged counter ion, such as Cl^{⊖}; F^{⊖}; Br^{⊖}; CF₃SO₃^{⊖}; PCl₆^{⊖}; PF₆^{⊖}; FeCl₄^{⊖}; Cl₃^{⊖}; PO₂Cl₂^{⊖}; ClHCl^{⊖}; Cl(SO₃)₂^{⊖}; ClSO₃^{⊖}; CH₃OSO₃^{⊖}; BF₄^{⊖}; NO₃^{⊖}; SbCl₆^{⊖}; C₂H₅OSO₃^{⊖}; HSO₄^{⊖}; H₂PO₄^{⊖}; CH₃COO^{⊖}; CH₃SO₃^{⊖}; and NO₂^{⊖}.

Generally, the reaction of the compound of Formula IA with the compound of Formula IB is performed in a reaction medium that includes a solvent, preferably an organic aprotic solvent. One preferred solvent is CH₂Cl₂.

The reaction medium can further include a base. In some embodiments, the base is an amine, such as a trialkyl amine, for example triethyl amine.

The reaction of the compound of Formula IA with the compound of Formula IB can be performed at a variety of temperatures. Preferably, the reaction is performed at a temperature of about −20° C. or greater, for example at a temperature of from about −20° C. to about 20° C.

In some embodiments, R¹ and R² are each independently alkyl or substituted alkyl. In some such embodiments, R¹ is methyl and R² is propyl.

In some embodiments, R³ is alkyl or substituted alkyl. In some such embodiments, R³ is —CH₂C(CH₃)₂OH or —CH₂CH(CH₃)₂.

In some embodiments, R⁴ and R⁵ taken together form a heteroaryl or substituted heteroaryl group. In some embodiments, R⁴ and R⁵ taken together form a quinolinyl or substituted quinolinyl group. In some further embodiments, R⁴ and R⁵ taken together form a pyridyl or substituted pyridyl group. In some further embodiments, R⁴ and R⁵ taken together form a heteroaryl group substituted with a halogen, amino, or substituted amino group.

In some embodiments of the methods of the invention, R⁴ and R⁵ taken together form a heteroaryl group substituted with a halogen; and the methods further include the step of displacing the halogen with an amino or substituted amino group, to form a compound wherein R⁴ and R⁵ taken together form a heteroaryl group substituted with an amino or substituted amino group. In a more particular embodiment the halogen is displaced with an azide or protected amino group. In a more particular embodiment thereof said azide is converted to a primary amino group. In another more particular embodiment thereof said protected amino group is deprotected to form a primary amino group.

In a second aspect, the invention provides methods for synthesizing a compound of Formula II:

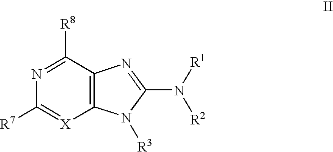

the method comprising the step of:

reacting a compound of Formula IA:

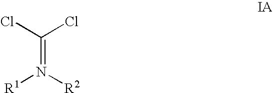

with a compound of Formula IIB:

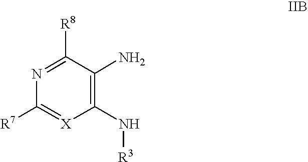

wherein,

X is N or CR⁶;

R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, hydroxy, alkoxy, substituted alkoxy, substituted alkyl, amino, substituted amino, acyl, and substituted carbonyl;

R⁶ and R⁷ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;

or R⁶ and R⁷ taken together form an aryl, substituted aryl, heteroaryl, substituted heteroaryl, cycloalkyl, substituted cycloalkyl, heterocyclyl, or substituted heterocyclyl group; and

R⁸ is selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some such embodiments, the compound of Formula IA further includes a negatively charged counter ion, such as Cl^{⊖}; F^{⊖}; Br^{⊖}; CF₃SO₃^{⊖}; PCl₆^{⊖}; PF₆^{⊖}; FeCl₄^{⊖}; Cl₃^{⊖}; PO₂Cl₂^{⊖}; ClHCl^{⊖}; Cl(SO₃)₂^{⊖}; ClSO₃^{⊖}; CH₃OSO₃^{⊖}; BF₄^{⊖}; NO₃^{⊖}; SbCl₆^{⊖}; C₂H₅OSO₃^{⊖}; HSO₄^{⊖}; H₂PO₄^{⊖}; CH₃COO^{⊖}; CH₃SO₃^{⊖}; and NO₂^{⊖}.

Generally, the reaction of the compound of Formula IA with the compound of Formula IB is performed in a reaction medium that includes an organic aprotic solvent. One preferred solvent is CH₂Cl₂.

Generally, the reaction medium can further include a base. In some embodiments, the base is an amine, such as a trialkyl amine, for example triethyl amine.

The reaction of the compound of Formula IA with the compound of Formula IB can be performed at a variety of temperatures. Preferably, the reaction is performed at a temperature of about −20° C. or greater, for example at a temperature of from about −20° C. to about 20° C.

In some embodiments, R¹ and R² are each independently alkyl or substituted alkyl. In some such embodiments, R¹ is methyl and R² is propyl. In some embodiments, R³ is alkyl or substituted alkyl. In some such embodiments, R³ is —CH₂C(CH₃)₂OH or —CH₂CH(CH₃)₂.

In some embodiments of the second aspect of the invention, X is CR⁶. In some such embodiments, R⁶ and R⁷ taken together form a phenyl or substituted phenyl group; or R⁶ and R⁷ taken together form a pyridyl or substituted pyridyl group; or R⁶ and R⁷ are each independently selected from the group consisting of hydrogen, alkyl, substituted alkyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some embodiments, R⁸ is a halogen, amino, or substituted amino group. In some such embodiments, R⁸ is a di-p-methoxybenzyl)amino group (i.e., —N(PMB)₂), and the methods further include the step of removing the p-methoxybenzyl (PMB) groups from the —N(PMB)₂ group, providing a compound wherein R⁸ is an amino (—NH₂) group. In other embodiments R⁸ is a halogen and is subsequently reacted with sodium azide. In a more particular embodiment R⁸ is —N₃ and the methods further comprise converting the —N₃ (azide) to an amino group.

In some embodiments, R⁸ is a halogen, and the methods further include the step of displacing the halogen with an amino or substituted amino group, to form a compound wherein R⁸ is an amino or substituted amino group. In a more particular embodiment the halogen is displaced with an azide or protected amino group. In a more particular embodiment thereof said azide is converted to a primary amino group. In another more particular embodiment thereof said protected amino group is deprotected to form a primary amino group.

In some embodiments, R⁸ is hydrogen, and the methods further include the step of reacting the compound of Formula II with an oxidizing agent to form an N-oxide (designated N→O) at the 5-position of the compound of Formula II. Suitable oxidizing agents are known in the art, and include, for example, metachloroperoxybenzoic acid (mCPBA) and hydrogen peroxide (H₂O₂). In some embodiments, the N-oxide is further reacted with a halogenating agent, to form a compound wherein R⁸ is a halogen, for example chlorine. Suitable halogenating agents are known in the art, and include, for example, POCl₃.

In some embodiments, the compound of Formula IIB:

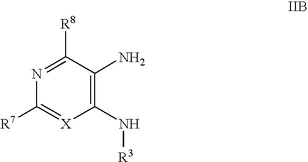

can be prepared by reacting a compound of Formula IIC:

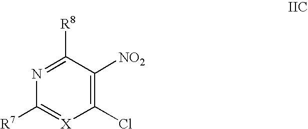

with a compound of formula H₂N—R³, to form a compound of Formula IID:

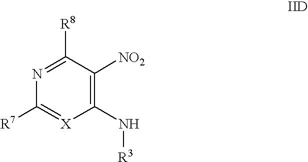

and reacting the compound of Formula IID with a hydrogenating agent. In some such embodiments, R⁸ is a halogen, preferably chlorine.

The reaction of the compounds of Formula IIC and H₂N—R³ to form a compound of Formula IID is preferably performed in a reaction medium that contains a solvent, preferably an aprotic organic solvent. One suitable solvent is N-methylpyrrolidinone (NMP). The reaction can be performed at a variety of temperatures, including room temperature (i.e., about 25° C.).

The reduction of the nitro group of the compound of Formula IID to an amine group (also referred to as the reaction of the compound of Formula IID with the hydrogenating agent) can be performed by any of a variety of reagents known to be useful to reduce nitro groups to amino groups. Two suitable reagents for the reactions are dithionate in acetone/water, and Zn dust in NH₄OH/methanol. As the reaction tends to be exothermic, it is preferred that the reaction be performed with cooling.

In some embodiments, the compound of Formula IIC:

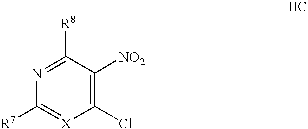

wherein R⁸ is chloro, can be prepared by reacting a compound of Formula IIE:

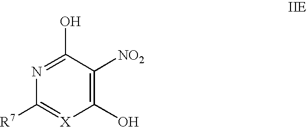

with a chlorinating agent. A variety of chlorinating agents, as are known in the art, are suitable for use in the reaction. One preferred chlorinating agent is PhPOCl₂. Generally, the reaction of the compound of Formula IIE and the chlorinating agent can be performed at a variety of temperatures, preferably from about 50° C. to about 150° C.

In some embodiments, the compound of Formula IIE can be prepared by reacting a compound of Formula IIF:

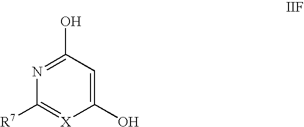

with a nitrosylating agent. One preferred nitrosylating agent is HNO₃, preferably in acetic acid. The nitrosylation reaction can be performed at a variety of temperatures, for example at a temperature of from about 50° C. to about 150° C.

In a third aspect, the invention provides methods for synthesizing a compound of Formula III:

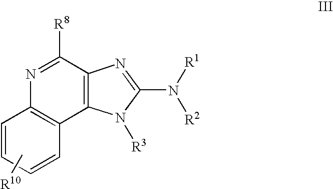

comprising:

reacting a compound of Formula IA:

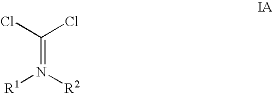

with a compound of Formula IIIB:

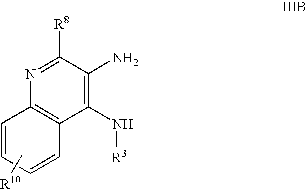

wherein:

R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, hydroxy, substituted alkyl, alkoxy, substituted alkoxy, amino, substituted amino, acyl, and substituted carbonyl;

R⁸ and R¹⁰ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some such embodiments, the compound of Formula IA further includes a negatively charged counter ion, such as Cl^{⊖}; F^{⊖}; Br^{⊖}; CF₃SO₃^{⊖}; PCl₆^{⊖}; PF₆^{⊖}; FeCl₄^{⊖}; Cl₃^{⊖}; PO₂Cl₂^{⊖}; ClHCl^{⊖}; Cl(SO₃)₂^{⊖}; ClSO₃^{⊖}; CH₃OSO₃^{⊖}; BF₄^{⊖}; NO₃^{⊖}; SbCl₆^{⊖}; C₂H₅OSO₃^{⊖}; HSO₄^{⊖}; H₂PO₄^{⊖}; CH₃COO^{⊖}; CH₃SO₃^{⊖}; and NO₂^{⊖}.

Generally, the reaction of the compound of Formula IA with the compound of Formula IIIB is performed in a reaction medium that includes a solvent, preferably an organic aprotic solvent. One preferred solvent is CH₂Cl₂.

The reaction medium can further include a base. In some embodiments, the base is an amine, such as a trialkyl amine, for example triethyl amine.

The reaction of the compound of Formula IA with the compound of Formula IIIB can be performed at a variety of temperatures. Preferably, the reaction is performed at a temperature of about −20° C. or greater, for example at a temperature of from about −20° C. to about 20° C.

In some embodiments, R¹ and R² are each independently alkyl or substituted alkyl. In some such embodiments, R¹ is methyl and R² is propyl.

In some embodiments, R³ is alkyl or substituted alkyl. In some such embodiments, R³ is —CH₂C(CH₃)₂OH or —CH₂CH(CH₃)₂.

In some embodiments, R¹⁰ is H.

In some further embodiments, R⁸ is a halogen, hydrogen, amino, or substituted amino group. In some such embodiments, R⁸ is a —N(PMB)₂ group, and the methods further include the step of removing the p-methoxybenzyl (PMB) groups from the —N(PMB)₂ group, providing a compound wherein R⁸ is an amino (—NH₂) group.

In some embodiments, R⁸ is a halogen, and the methods further include the step of displacing the halogen with an amino or substituted amino group, to form a compound wherein R⁸ is an amino or substituted amino group.

In some embodiments, R⁸ is hydrogen, and the methods further include the step of reacting the compound of Formula III with an oxidizing agent to form an N-oxide (designated N→O) at the 5-position of the compound of Formula III. Suitable oxidizing agents are known in the art, and include, for example, metachloroperoxybenzoic acid (mCPBA) and hydrogen peroxide (H₂O₂). In some embodiments, the N-oxide is further reacted with a halogenating agent, for example chlorine, to form a compound wherein R⁸ is a halogen and X is N. Suitable halogenating agents are known in the art, and include, for example, POCl₃. In some embodiments, the methods further include displacing the halogen R⁸ with an amino or substituted amino group, to form a compound wherein R⁸ is an amino or substituted amino group.

In some embodiments, the compound of Formula IIIB:

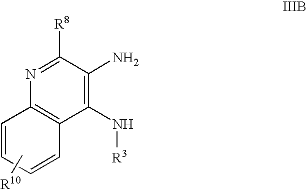

can be prepared by reacting a compound of Formula IIIC:

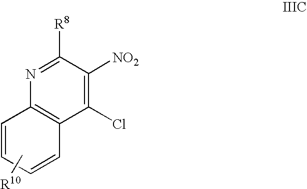

with a compound of formula H₂N—R³, to form a compound of Formula IIID:

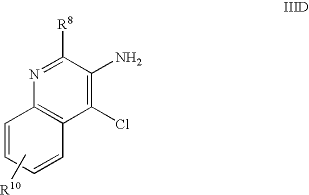

and reacting said compound of Formula IIID with a hydrogenating agent. In some embodiments, R⁸ is chlorine.

Generally, the reagents and conditions described above for the reaction of compounds of Formula IIC and H₂N—R³, to produce the compound of Formula IID, and subsequent hydrogenation of the compound of Formula IID, are applicable to the reaction of the compound of Formula IIIC and H₂N—R³, to produce the compound of formula IIID, and subsequent hydrogenation thereof.

In some embodiments, the compound of Formula IIIC, wherein R⁸ is chlorine, can be prepared by reacting a compound of Formula IIIE:

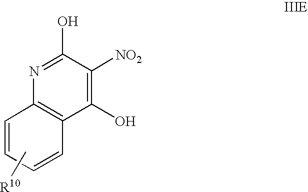

with a chlorinating agent. Generally, the reagents and conditions described above for the reaction of compounds of Formula IIE and the chlorinating agent, are applicable to the reaction of the compound of Formula IIIC and the chlorinating agent.

In some embodiments, the compound of Formula IIIE can be prepared by reacting a compound of Formula IIF:

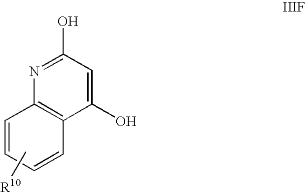

with a nitrosylating agent. One preferred nitrosylating agent is HNO₃, preferably in acetic acid. The nitrosylation reaction can be performed at a variety of temperatures, for example at a temperature of from about 50° C. to about 150° C.

In some embodiments of the methods of the invention, the compound of Formula IA:

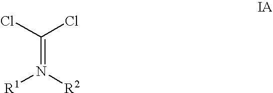

can be prepared by reacting a compound of Formula IC:

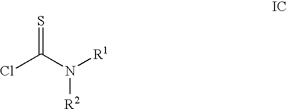

with phosgene or diphosgene.

In a fourth aspect, the invention provides methods for the preparation of a compound of Formula II:

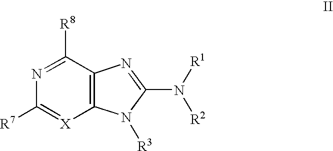

comprising:

reacting a compound of Formula ID:

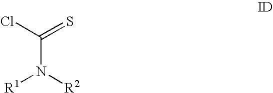

with a compound of Formula IIB:

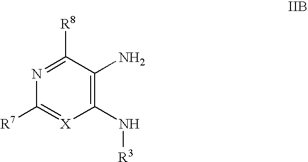

wherein,

X is N or CR⁶;

R¹ and R² are each independently selected from the group consisting of hydrogen, alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, hydroxy, alkoxy, substituted alkoxy, substituted alkyl, amino, substituted amino, acyl, and substituted carbonyl;

R⁶ and R⁷ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;

or R⁶ and R⁷ taken together form an aryl, substituted aryl, heteroaryl, substituted heteroaryl, cycloalkyl, substituted cycloalkyl, heterocyclyl, or substituted heterocyclyl group; and

R⁸ is selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

Generally, the reaction of the compound of Formula IA with the compound of Formula IB is performed in a reaction medium that includes a solvent, preferably an organic aprotic solvent. One preferred solvent is CH₂Cl₂.

The reaction medium can further include a base. In some embodiments, the base is Na₂CO₃. In some embodiments, the reaction medium further comprises Hg(OAc)₂.

The reaction of the compound of Formula ID with the compound of Formula IIB can be performed at a variety of temperatures. Preferably, the reaction is performed at a temperature of about −79° C. or greater, for example at a temperature of from about −79° C. to about 25° C.

In some embodiments, R¹ and R² are both independently alkyl or substituted alkyl. In some embodiments, R¹ is methyl R² is propyl.

In some embodiments, R³ is alkyl or substituted alkyl, for example —CH₂C(CH₃)₂OH or —CH₂CH(CH₃)₂.

In some embodiments, X is CR⁶. In some such embodiments, R⁶ and R⁷ taken together form a phenyl or substituted phenyl group; or R⁶ and R⁷ taken together form a pyridyl or substituted pyridyl group; or R⁶ and R⁷ are each independently selected from the group consisting of hydrogen, alkyl, substituted alkyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some embodiments, R⁸ is a substituted amino group, for example a —N(PMB)₂ group. In some such embodiments, the methods further include the step of removing the PMB groups from the nitrogen of the R⁸ group to form a compound wherein R₈ is an amino group.

In some embodiments, R⁸ is a halogen, and the methods further include the step of displacing the halogen with an amino or substituted amino group, to form a compound wherein R⁸ is an amino or substituted amino group.

In some embodiments, R⁸ is hydrogen, and the methods further include the step of reacting the compound of Formula II with an oxidizing agent to form an N-oxide (designated N→O) at the 5-position of the compound of Formula II. Suitable oxidizing agents are known in the art, and include, for example, metachloroperoxybenzoic acid (mCPBA) and hydrogen peroxide (H₂O₂). In some embodiments, the N-oxide is further reacted with a halogenating agent, to form a compound wherein R⁸ is a halogen, for example chlorine. Suitable halogenating agents are known in the art, and include, for example, POCl₃.

In some embodiments, the compound of Formula IIB:

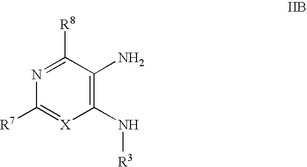

can be prepared by reacting a compound of Formula IIC:

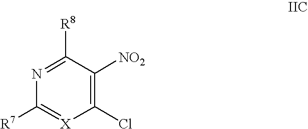

with a compound of formula H₂N—R³, to form a compound of Formula IID:

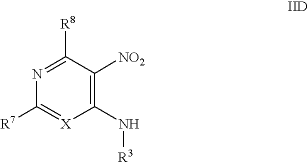

and reacting the compound of Formula IID with a hydrogenating agent. In some embodiments, R⁸ is a halogen. In some such embodiments, the methods further include the step of reacting the compound of Formula IID with HN(PMB)₂, to form a compound wherein R⁸ is —N(PMB)₂. In some further embodiments wherein R⁸ is a halogen, the compound of Formula IIC, wherein R⁸ is chlorine, can be prepared by reacting a compound of Formula IIE:

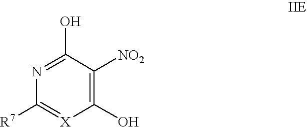

with a chlorinating agent, as described above. In some embodiments, the chlorinating agent is PhPOCl₂. Generally, the reagents and conditions are as described above the reaction of the compound of Formula IIE and the chlorinating agent.

In some embodiments, the compound of Formula IIE:

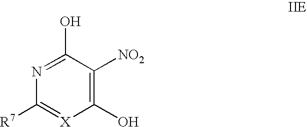

can be prepared by reacting a compound of Formula IIF:

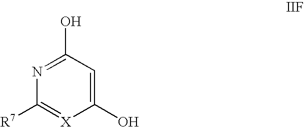

with a nitrosylating agent. One preferred nitrosylating agent HNO₃, preferably in acetic acid. The nitrosylation can be performed at a variety of temperatures, for example at a temperature of from about 50° C. to about 150° C.

In a fourth aspect, the invention provides methods for preparing a compound of Formula III:

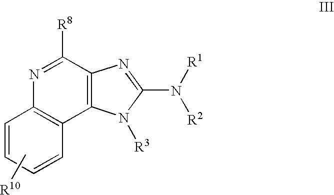

comprising:

reacting a compound of Formula ID:

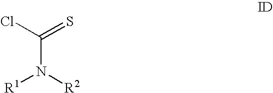

with a compound of Formula IIIB:

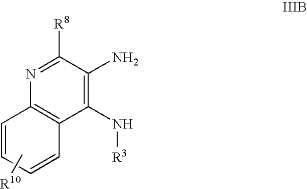

wherein:

R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;

or R¹ and R² taken together form a heterocyclyl or substituted heterocyclyl group;

R³ is selected from the group consisting of H, alkyl, hydroxy, substituted alkyl, alkoxy, substituted alkoxy, amino, substituted amino, carbonyl, and substituted carbonyl;

R⁸ and R¹⁰ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

Generally, the reaction of the compound of Formula ID with the compound of Formula IIIB is performed in a reaction medium that includes a solvent, preferably an organic aprotic solvent. One preferred solvent is CH₂Cl₂.

The reaction medium can further include a base. In some embodiments, the base is Na₂CO₃. In some embodiments, the reaction medium further comprises Hg(OAc)₂.

The reaction of the compound of Formula ID with the compound of Formula IIB can be performed at a variety of temperatures. Preferably, the reaction is performed at a temperature of about −79° C. or greater, for example at a temperature of from about −79° C. to about 25° C.

In some embodiments, R¹ and R² are both independently alkyl or substituted alkyl. In some embodiments, R¹ is methyl and R² is propyl.

In some embodiments, R³ is alkyl or substituted alkyl, for example —CH₂C(CH₃)₂OH or —CH₂CH(CH₃)₂.

In some embodiments, X is CR⁶. In some such embodiments, R⁶ and R⁷ taken together form a phenyl or substituted phenyl group; or R⁶ and R⁷ taken together form a pyridyl or substituted pyridyl group; or R⁶ and R⁷ are each independently selected from the group consisting of hydrogen, alkyl, substituted alkyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

In some embodiments, R⁸ is a substituted amino group, for example a —N(PMB)₂ group. In some such embodiments, the methods further include the step of removing the PMB groups from the nitrogen of the R⁸ group to form a compound wherein R₈ is an amino group.

In some embodiments, R⁸ is a halogen, and the methods further include the step of displacing the halogen with an amino or substituted amino group, to form a compound wherein R⁸ is an amino or substituted amino group.

In some embodiments, R⁸ is hydrogen, and the methods further include the step of reacting the compound of Formula III with an oxidizing agent to form an N-oxide (designated N→O) at the 5-position of the compound of Formula III. Suitable oxidizing agents are known in the art, and include, for example, metachloroperoxybenzoic acid (mCPBA) and hydrogen peroxide (H₂O₂). In some embodiments, the N-oxide is further reacted with a halogenating agent, to form a compound wherein R⁸ is a halogen, for example chlorine. Suitable halogenating agents are known in the art, and include, for example, POCl₃.

In some embodiments, the compound of Formula IIIB:

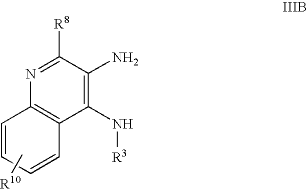

can be prepared by reacting a compound of Formula IIIC:

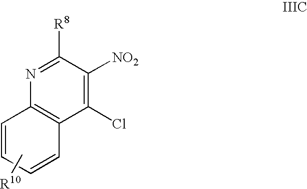

with a compound of formula H₂N—R³, to form a compound of Formula IIID:

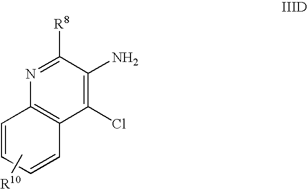

and reacting the compound of Formula IIID with a hydrogenating agent. In some embodiments, R⁸ is a halogen. In some such embodiments, the methods further include the step of reacting the compound of Formula IIID with HN(PMB)₂, to form a compound wherein R⁸ is —N(PMB)₂. In some further embodiments wherein R⁸ is a halogen, the compound of Formula IIIC, wherein R⁸ is chlorine, can be prepared by reacting a compound of Formula IIIE:

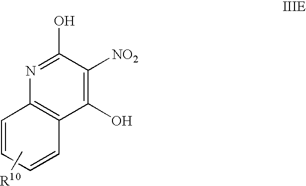

with a chlorinating agent, as described above. In some embodiments, the chlorinating agent is PhPOCl₂. Generally, the reagents and conditions are as described above the reaction of the compound of Formula IIE and the chlorinating agent.

In some embodiments, the compound of Formula IIIE:

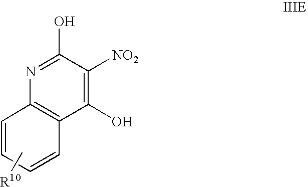

can be prepared by reacting a compound of Formula IIIF:

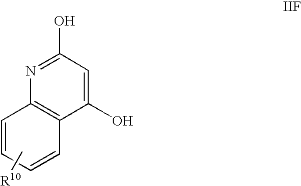

with a nitrosylating agent. One preferred nitrosylating agent HNO₃, preferably in acetic acid. The nitrosylation can be performed at a variety of temperatures, for example at a temperature of from about 50° C. to about 150° C.

The present invention further provides methods of inducing an immune response in a subject, comprising administering a compound prepared according to any of the methods disclosed herein, to the subject in an amount sufficient to induce an immune response in the subject. In some embodiments, the immune response is TLR7 and/or TLR8 related.

Additional embodiments, methods and compositions contemplated to be useful in the instant invention are disclosed in PCT/US2005/032721, PCT/US2005/022769, PCT/US2005/022520 and U.S. Ser. Nos. 10/814,480, 10/762,873, 60/582,654, 10/405,495, and 10/748,071 which are each hereby incorporated by reference in their entireties and for all purposes as if set forth fully herein.

Generally, a SMIP or a composition comprising a SMIP is considered effective to elicit an immune response at a concentration of 300 μM or less in some embodiments, 200 μM or less in some embodiments, 100 μM or less in some embodiments, or 20 μM or less in some embodiments if the SMIP compound effects (a) the production of TNF-α in an in vitro cell based assay of human peripheral blood mononuclear cells, and (b) a concentration of human peripheral blood mononuclear cells (PBMCs) of about 500,000/mL, when the cells are exposed to the compound for a period of about 18-24 hours, preferably about 24 hours.

The above method of stimulating a local immune response, for example in selected cells or tissues of a patient, includes the stimulation of a local immune response where the selected cells or tissues are infected or cancerous. In some embodiments, the selected cells or tissues are infected with a fungus or bacterium. In some embodiments, the selected tissues are inflamed with an allergen, for example in an asthmatic condition. In other embodiments, the selected cells are infected with a virus or bacteria.

Another embodiment provides a method of inducing interferon biosynthesis in a subject. Such methods include administering a compound synthesized according to the methods described herein to the subject in an amount sufficient to induce interferon biosynthesis. In some such methods, a vaccine adjuvant of formula I is administered to the subject in an amount sufficient to induce interferon biosynthesis.

Another embodiment provides a compound synthesized according to the methods described herein, wherein the compound is co-administered with another agent to a patient in need thereof. In some such embodiments, the agent is an antigen or a vaccine. In embodiments, where the compound synthesized according to the methods described herein is co-administered to a patient or subject along with another agent, the compound synthesized according to the methods described herein may be administered to the subject before, during, or after the other agent is administered to the subject. Therefore, in some embodiments, the compound synthesized according to the methods described herein is administered to the subject at the same time that the other agent is administered to the subject.

Another embodiment provides a method of modulating an immune response in a subject. Such methods include administering a compound synthesized according to the methods described herein to the subject.

Another embodiment provides a method for inducing the production of TNF-α in a subject. Such methods include administering a compound synthesized according to the methods described herein to a subject in an amount sufficient to induce the production of TNF-α. In some such embodiment thereof, the compound has an average steady state drug concentration in the blood of less than 20 μM.

Another embodiment provides a method of inducing an immune response in a subject. The embodiment includes administering a compound synthesized according to the methods described herein to the subject in an amount sufficient to induce an immune response. In some such embodiments, the immune response involves the production of cytokines or increased production of TNF-α.

Another embodiment provides a method of inducing an immune response in a subject suffering from a microbial infection. The method includes administering a compound synthesized according to the methods described herein to the subject in an amount sufficient to induce an immune response.

Another embodiment provides a method of inducing an immune response in a subject suffering from a viral infection or a disease condition caused by a virus. The method includes administering a compound synthesized according to the methods described herein to the subject in an amount sufficient to induce an immune response in the subject. In some such embodiments, the subject is suffering from a viral infection or disease condition caused by the hepatitis C virus (HCV). In other embodiments, the subject is suffering from a viral infection or disease condition caused by the human immunodeficiency virus (HIV). In another embodiment or method, the compound synthesized according to the methods described herein is administered topically to a subject.

Another embodiment provides a method of inducing an immune response in a subject for prevention of a viral infection or a disease condition caused by a virus. The method includes administering a compound synthesized according to the methods described herein to the subject in an amount sufficient to induce an immune response in the subject. In some such embodiments, the subject is prevented from a viral infection or disease condition. In other embodiments, the subject is protected from a microbial or other pathogenic infection, such as a those described herein.

Another embodiment provides a method of inducing an immune response in a subject suffering from an abnormal cellular proliferation or cancer. The method includes administering a compound synthesized according to the methods described herein to the subject in an amount sufficient to induce an immune response. In some embodiments, the compound is administered to a subject that is suffering from a disease associated with abnormal cellular proliferation. In some such embodiments, the disease is selected from neuro-fibromatosis, atherosclerosis, pulmonary fibrosis, arthritis, psoriasis, glomerulonephritis, restenosis, proliferative diabetic retinopathy (PDR), hypertrophic scar formation, inflammatory bowel disease, transplantation rejection, angiogenesis, or endotoxic shock.

Other embodiments provide methods of inducing an immune response in a subject suffering from an allergic disease. Such methods include administering a compound synthesized according to the methods described herein to the subject in an amount sufficient to induce an immune response.

Another embodiment provides a method of inducing an immune response in a subject suffering from asthma. The method includes administering a compound synthesized according to the methods described herein to the subject in an amount sufficient to induce an immune response. In some embodiments, asthma may be treated by steering the immune response away from Type 2 cytokine secretion and effector mechanism (e.g., IgE production and/or mast cell/basophil activation).

Another embodiment provides a method of inducing an immune response in a subject suffering from precancerous lesions. The method includes administering a compound synthesized according to the methods described herein to the subject in an amount sufficient to induce an immune response. In some such embodiments, the precancerous lesions are actinic keratosis. In other embodiments, the precancerous lesions are selected from actinic keratosis, atypical or dysplastic nevi, or premalignant lentigos. In another embodiment or method, the compound synthesized according to the methods described herein is administered topically to a subject.

Other embodiments provide a method of inhibiting a kinase in a subject. Such methods include administering the compound synthesized according to the methods described herein to the subject.

Another embodiment provides a method of modulating an immune response in a subject. The method includes administering a compound synthesized according to the methods described herein to the subject in an amount sufficient to inhibit a kinase in the subject. In some such embodiments, the kinase is selected from EGFr, c-Kit, bFGF, Kdr, CHK1, CDK, cdc-2, Akt, PDGF, PI3K, VEGF, PKA, PKB, src, c-Met, Abl, Ras, RAF, MEK, or combinations thereof. In another embodiment or method, the compound synthesized according to the methods described herein is administered topically to a subject.

Another embodiment provides a method of inducing an immune response in a subject, comprising: administering to the subject a compound synthesized according to the methods described herein and an antigen, wherein the compound induces or enhances an immune response to the antigen in the subject. More particularly the antigen is influenza or any other antigen described herein.

### Antigens:

Compositions of the invention may be administered in conjunction with one or more antigens for use in therapeutic, prophylactic, or diagnostic methods of the present invention. Preferred antigens include those listed below. Additionally, the compositions of the present invention may be used to treat or prevent infections caused by any of the below-listed pathogens. In addition to combination with the antigens described below, the compositions of the invention may also be combined with an adjuvant as described herein.

Antigens for use with the invention include, but are not limited to, one or more of the following antigens set forth below, or antigens derived from one or more of the pathogens set forth below:

### A. Bacterial Antigens

Bacterial antigens suitable for use in the invention include proteins, polysaccharides, lipopolysaccharides, and outer membrane vesicles which may be isolated, purified or derived from a bacteria. In addition, bacterial antigens may include bacterial lysates and inactivated bacteria formulations. Bacteria antigens may be produced by recombinant expression. Bacterial antigens preferably include epitopes which are exposed on the surface of the bacteria during at least one stage of its life cycle. Bacterial antigens are preferably conserved across multiple serotypes. Bacterial antigens include antigens derived from one or more of the bacteria set forth below as well as the specific antigens examples identified below.

Neisseria meningitides: Meningitides antigens may include proteins (such as those identified in References 1-7), saccharides (including a polysaccharide, oligosaccharide or lipopolysaccharide), or outer-membrane vesicles (References 8, 9, 10, 11) purified or derived from N. meningitides serogroup such as A, C, W135, Y, and/or B. Meningitides protein antigens may be selected from adhesions, autotransporters, toxins, Fe acquisition proteins, and membrane associated proteins (preferably integral outer membrane protein).

Streptococcus pneumoniae: Streptococcus pneumoniae antigens may include a saccharide (including a polysaccharide or an oligosaccharide) and/or protein from Streptococcus pneumoniae. Saccharide antigens may be selected from serotypes 1, 2, 3, 4, 5, 6B, 7F, 8, 9N, 9V, 10A, 11A, 12F, 14, 15B, 17F, 18C, 19A, 19F, 20, 22F, 23F, and 33F. Protein antigens may be selected from a protein identified in WO 98/18931, WO 98/18930, U.S. Pat. No. 6,699,703, U.S. Pat. No. 6,800,744, WO 97/43303, and WO 97/37026. Streptococcus pneumoniae proteins may be selected from the Poly Histidine Triad family (PhtX), the Choline Binding Protein family (CbpX), CbpX truncates, LytX family, LytX truncates, CbpX truncate-LytX truncate chimeric proteins, pneumolysin (Ply), PspA, PsaA, Sp128, Sp101, Sp130, Spl25 or Spl33.

Streptococcus pyogenes (Group A Streptococcus): Group A Streptococcus antigens may include a protein identified in WO 02/34771 or WO 2005/032582 (including GAS 40), fusions of fragments of GAS M proteins (including those described in WO 02/094851, and Dale, Vaccine (1999) 17:193-200, and Dale, Vaccine 14(10): 944-948), fibronectin binding protein (Sfb1), Streptococcal heme-associated protein (Shp), and Streptolysin S (SagA).

Moraxella catarrhalis: Moraxella antigens include antigens identified in WO 02/18595 and WO 99/58562, outer membrane protein antigens (HMW-OMP), C-antigen, and/or LPS.

Bordetella pertussis: Pertussis antigens include pertussis holotoxin (PT) and filamentous haemagglutinin (FHA) from B. pertussis, optionally also combination with pertactin and/or agglutinogens 2 and 3 antigen.

Staphylococcus aureus: Staph aureus antigens include S. aureus type 5 and 8 capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin).

Staphylococcus epidermis: S. epidermidis antigens include slime-associated antigen (SAA).

Clostridium tetani (Tetanus): Tetanus antigens include tetanus toxoid (TT), preferably used as a carrier protein in conjunction/conjugated with the compositions of the present invention.

Cornynebacterium diphtheriae (Diphtheria): Diphtheria antigens include diphtheria toxin, preferably detoxified, such as CRM₁₉₇. Additionally antigens capable of modulating, inhibiting or associated with ADP ribosylation are contemplated for combination/co-administration/conjugation with the compositions of the present invention. The diphtheria toxoids may be used as carrier proteins.

Haemophilus influenzae B (Hib): Hib antigens include a Hib saccharide antigen.

Pseudomonas aeruginosa: Pseudomonas antigens include endotoxin A, Wzz protein, P. aeruginosa LPS, more particularly LPS isolated from PAO1 (O5 serotype), and/or Outer Membrane Proteins, including Outer Membrane Proteins F (OprF) (Infect Immun. 2001 May; 69(5): 3510-3515).

Legionella pneumophila. Bacterial antigens may be derived from Legionella pneumophila.

Streptococcus agalactiae (Group B Streptococcus): Group B Streptococcus antigens include a protein or saccharide antigen identified in WO 02/34771, WO 03/093306, WO 04/041157, or WO 2005/002619 (including proteins GBS 80, GBS 104, GBS 276 and GBS 322, and including saccharide antigens derived from serotypes Ia, Ib, Ia/c, II, III, IV, V, VI, VII and VIII).

Neiserria gonorrhoeae: Gonorrhoeae antigens include Por (or porin) protein, such as PorB (see Zhu et al., Vaccine (2004) 22:660-669), a transferring binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al., J Infectious Disease (2000) 182:848-855), also see e.g. WO99/24578, WO99/36544, WO99/57280, WO02/079243).

Chlamydia trachomatis: Chlamydia trachomatis antigens include antigens derived from serotypes A, B, Ba and C (agents of trachoma, a cause of blindness), serotypes L₁, L₂ & L₃ (associated with Lymphogranuloma venereum), and serotypes, D-K. Chlamydia trachomas antigens may also include an antigen identified in WO 00/37494, WO 03/049762, WO 03/068811, or WO 05/002619, including PepA (CT045), LcrE (CT089), ArtJ (CT381), DnaK (CT396), CT398, OmpH-like (CT242), L7/L12 (CT316), OmcA (CT444), AtosS (CT467), CT547, Eno (CT587), HrtA (CT823), and MurG (CT761).

Treponema pallidum (Syphilis): Syphilis antigens include TmpA antigen.

Haemophilus ducreyi (causing chancroid): Ducreyi antigens include outer membrane protein (DsrA).

Enterococcus faecalis or Enterococcus faecium: Antigens include a trisaccharide repeat or other Enterococcus derived antigens provided in U.S. Pat. No. 6,756,361.

Helicobacter pylori: H pylori antigens include Cag, Vac, Nap, HopX, HopY and/or urease antigen.

Staphylococcus saprophyticus: Antigens include the 160 kDa hemagglutinin of S. saprophyticus antigen.

Yersinia enterocolitica Antigens include LPS (Infect Immun. 2002 August; 70(8): 4414).

E. coli: E. coli antigens may be derived from enterotoxigenic E. coli (ETEC), enteroaggregative E. coli (EAggEC), diffusely adhering E. coli (DAEC), enteropathogenic E. coli (EPEC), and/or enterohemorrhagic E. coli (EHEC).

Bacillus anthracis (anthrax): B. anthracis antigens are optionally detoxified and may be selected from A-components (lethal factor (LF) and edema factor (EF)), both of which can share a common B-component known as protective antigen (PA).

Yersinia pestis (plague): Plague antigens include F1 capsular antigen (Infect Immun. 2003 January; 71(1)): 374-383, LPS (Infect Immun. 1999 October; 67(10): 5395), Yersinia pestis V antigen (Infect Immun. 1997 November; 65(11): 4476-4482).

Mycobacterium tuberculosis: Tuberculosis antigens include lipoproteins, LPS, BCG antigens, a fusion protein of antigen 85B (Ag85B) and/or ESAT-6 optionally formulated in cationic lipid vesicles (Infect Immun. 2004 October; 72(10): 6148), Mycobacterium tuberculosis (Mtb) isocitrate dehydrogenase associated antigens (Proc Natl Acad Sci USA. 2004 Aug. 24; 101(34): 12652), and/or MPT51 antigens (Infect Immun. 2004 July; 72(7): 3829).

Rickettsia: Antigens include outer membrane proteins, including the outer membrane protein A and/or B (OmpB) (Biochim Biophys Acta. 2004 Nov. 1; 1702(2):145), LPS, and surface protein antigen (SPA) (J Autoimmun. 1989 June; 2 Suppl:81).

Listeria monocytogenes. Bacterial antigens may be derived from Listeria monocytogenes.

Chlamydia pneumoniae: Antigens include those identified in WO 02/02606.

Vibrio cholerae: Antigens include proteinase antigens, LPS, particularly lipopolysaccharides of Vibrio cholerae II, O1 Inaba O-specific polysaccharides, V. cholera O139, antigens of IEM108 vaccine (Infect Immun. 2003 October; 71(10):5498-504), and/or Zonula occludens toxin (Zot).

Salmonella typhi (typhoid fever): Antigens include capsular polysaccharides preferably conjugates (Vi, i.e. vax-TyVi).

Borrelia burgdorferi (Lyme disease): Antigens include lipoproteins (such as OspA, OspB, Osp C and Osp D), other surface proteins such as OspE-related proteins (Erps), decorin-binding proteins (such as DbpA), and antigenically variable VI proteins, such as antigens associated with P39 and P13 (an integral membrane protein, Infect Immun. 2001 May 69(5): 3323-3334), VlsE Antigenic Variation Protein (J Clin Microbiol. 1999 December; 37(12): 3997).

Porphyromonas gingivalis: Antigens include P. gingivalis outer membrane protein (OMP).

Klebsiella: Antigens include an OMP, including OMP A, or a polysaccharide optionally conjugated to tetanus toxoid.

Further bacterial antigens of the invention may be capsular antigens, polysaccharide antigens or protein antigens of any of the above. Further bacterial antigens may also include an outer membrane vesicle (OMV) preparation. Additionally, antigens include live, attenuated, and/or purified versions of any of the aforementioned bacteria. The antigens of the present invention may be derived from gram-negative or gram-positive bacteria. The antigens of the present invention may be derived from aerobic or anaerobic bacteria.

Additionally, any of the above bacterial-derived saccharides (polysaccharides, LPS, LOS or oligosaccharides) can be conjugated to another agent or antigen, such as a carrier protein (for example CRM₁₉₇). Such conjugation may be direct conjugation effected by reductive amination of carbonyl moieties on the saccharide to amino groups on the protein, as provided in U.S. Pat. No. 5,360,897 and Can J Biochem Cell Biol. 1984 May; 62(5):270-5. Alternatively, the saccharides can be conjugated through a linker, such as, with succinamide or other linkages provided in Bioconjugate Techniques, 1996 and CRC, Chemistry of Protein Conjugation and Cross-Linking, 1993.

### B. Viral Antigens

Viral antigens suitable for use in the invention include inactivated (or killed) virus, attenuated virus, split virus formulations, purified subunit formulations, viral proteins which may be isolated, purified or derived from a virus, and Virus Like Particles (VLPs). Viral antigens may be derived from viruses propagated on cell culture or other substrate. Alternatively, viral antigens may be expressed recombinantly. Viral antigens preferably include epitopes which are exposed on the surface of the virus during at least one stage of its life cycle. Viral antigens are preferably conserved across multiple serotypes or isolates. Viral antigens include antigens derived from one or more of the viruses set forth below as well as the specific antigens examples identified below.

Orthomyxovirus: Viral antigens may be derived from an Orthomyxovirus, such as Influenza A, B and C. Orthomyxovirus antigens may be selected from one or more of the viral proteins, including hemagglutinin (HA), neuraminidase (NA), nucleoprotein (NP), matrix protein (M1), membrane protein (M2), one or more of the transcriptase components (PB1, PB2 and PA). Preferred antigens include HA and NA.

Influenza antigens may be derived from interpandemic (annual) flu strains. Alternatively influenza antigens may be derived from strains with the potential to cause pandemic a pandemic outbreak (i.e., influenza strains with new haemagglutinin compared to the haemagglutinin in currently circulating strains, or influenza strains which are pathogenic in avian subjects and have the potential to be transmitted horizontally in the human population, or influenza strains which are pathogenic to humans).

Paramyxoviridae viruses: Viral antigens may be derived from Paramyxoviridae viruses, such as Pneumoviruses (RSV), Paramyxoviruses (PIV) and Morbilliviruses (Measles).

Pneumovirus: Viral antigens may be derived from a Pneumovirus, such as Respiratory syncytial virus (RSV), Bovine respiratory syncytial virus, Pneumonia virus of mice, and Turkey rhinotracheitis virus. Preferably, the Pneumovirus is RSV. Pneumovirus antigens may be selected from one or more of the following proteins, including surface proteins Fusion (F), Glycoprotein (G) and Small Hydrophobic protein (SH), matrix proteins M and M2, nucleocapsid proteins N, P and L and nonstructural proteins NS1 and NS2. Preferred Pneumovirus antigens include F, G and M. See e.g., J Gen Virol. 2004 November; 85(Pt 11):3229). Pneumovirus antigens may also be formulated in or derived from chimeric viruses. For example, chimeric RSV/PIV viruses may comprise components of both RSV and PIV.

Paramyxovirus: Viral antigens may be derived from a Paramyxovirus, such as Parainfluenza virus types 1-4 (PIV), Mumps, Sendai viruses, Simian virus 5, Bovine parainfluenza virus and Newcastle disease virus. Preferably, the Paramyxovirus is PIV or Mumps. Paramyxovirus antigens may be selected from one or more of the following proteins: Hemagglutinin-Neuraminidase (HN), Fusion proteins F1 and F2, Nucleoprotein (NP), Phosphoprotein (P), Large protein (L), and Matrix protein (M). Preferred Paramyxovirus proteins include HN, F1 and F2. Paramyxovirus antigens may also be formulated in or derived from chimeric viruses. For example, chimeric RSV/PIV viruses may comprise components of both RSV and PIV. Commercially available mumps vaccines include live attenuated mumps virus, in either a monovalent form or in combination with measles and rubella vaccines (MMR).

Morbillivirus: Viral antigens may be derived from a Morbillivirus, such as Measles. Morbillivirus antigens may be selected from one or more of the following proteins: hemagglutinin (H), Glycoprotein (G), Fusion factor (F), Large protein (L), Nucleoprotein (NP), Polymerase phosphoprotein (P), and Matrix (M). Commercially available measles vaccines include live attenuated measles virus, typically in combination with mumps and rubella (MMR).

Picornavirus: Viral antigens may be derived from Picornaviruses, such as Enteroviruses, Rhinoviruses, Heparnavirus, Cardioviruses and Aphthoviruses. Antigens derived from Enteroviruses, such as Poliovirus are preferred.

Enterovirus: Viral antigens may be derived from an Enterovirus, such as Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 and Enterovirus 68 to 71. Preferably, the Enterovirus is poliovirus. Enterovirus antigens are preferably selected from one or more of the following Capsid proteins VP1, VP2, VP3 and VP4. Commercially available polio vaccines include Inactivated Polio Vaccine (IPV) and Oral poliovirus vaccine (OPV).

Heparnavirus: Viral antigens may be derived from an Heparnavirus, such as Hepatitis A virus (HAV). Commercially available HAV vaccines include inactivated HAV vaccine.

Togavirus: Viral antigens may be derived from a Togavirus, such as a Rubivirus, an Alphavirus, or an Arterivirus. Antigens derived from Rubivirus, such as Rubella virus, are preferred. Togavirus antigens may be selected from E1, E2, E3, C, NSP-1, NSPO-2, NSP-3 or NSP-4. Togavirus antigens are preferably selected from E1, E2 or E3. Commercially available Rubella vaccines include a live cold-adapted virus, typically in combination with mumps and measles vaccines (MMR).

Flavivirus: Viral antigens may be derived from a Flavivirus, such as Tick-borne encephalitis (TBE), Dengue (types 1, 2, 3 or 4), Yellow Fever, Japanese encephalitis, West Nile encephalitis, St. Louis encephalitis, Russian spring-summer encephalitis, Powassan encephalitis. Flavivirus antigens may be selected from PrM, M, C, E, NS-1, NS-2a, NS2b, NS3, NS4a, NS4b, and NS5. Flavivirus antigens are preferably selected from PrM, M and E. Commercially available TBE vaccine include inactivated virus vaccines.

Pestivirus: Viral antigens may be derived from a Pestivirus, such as Bovine viral diarrhea (BVDV), Classical swine fever (CSFV) or Border disease (BDV).

Hepadnavirus: Viral antigens may be derived from a Hepadnavirus, such as Hepatitis B virus. Hepadnavirus antigens may be selected from surface antigens (L, M and S), core antigens (HBc, HBe). Commercially available HBV vaccines include subunit vaccines comprising the surface antigen S protein.

Hepatitis C virus: Viral antigens may be derived from a Hepatitis C virus (HCV). (see, e.g. Hsu et al. (1999) Clin Liver Dis 3:901-915). HCV antigens may be selected from one or more of E1, E2, E1/E2, NS345 polyprotein, NS 345-core polyprotein, core, and/or peptides from the nonstructural regions (Houghton et al., Hepatology (1991) 14:381). For example, Hepatitis C virus antigens that may be used can include one or more of the following: HCV E1 and or E2 proteins, E1/E2 heterodimer complexes, core proteins and non-structural proteins, or fragments of these antigens, wherein the non-structural proteins can optionally be modified to remove enzymatic activity but retain immunogenicity (see, e.g. WO03/002065; WO01/37869 and WO04/005473).

Rhabdovirus: Viral antigens may be derived from a Rhabdovirus, such as a Lyssavirus (Rabies virus) and Vesiculovirus (VSV). Rhabdovirus antigens may be selected from glycoprotein (G), nucleoprotein (N), large protein (L), nonstructural proteins (NS). Commercially available Rabies virus vaccine comprise killed virus grown on human diploid cells or fetal rhesus lung cells.

Caliciviridae; Viral antigens may be derived from Calciviridae, such as Norwalk virus, and Norwalk-like Viruses, such as Hawaii Virus and Snow Mountain Virus.

Coronavirus: Viral antigens may be derived from a Coronavirus, SARS, Human respiratory coronavirus, Avian infectious bronchitis (IBV), Mouse hepatitis virus (MHV), and Porcine transmissible gastroenteritis virus (TGEV). Coronavirus antigens may be selected from spike (S), envelope (E), matrix (M), nucleocapsid (N), and Hemagglutinin-esterase glycoprotein (HE). Preferably, the Coronavirus antigen is derived from a SARS virus. SARS viral antigens are described in WO 04/92360;

Retrovirus: Viral antigens may be derived from a Retrovirus, such as an Oncovirus, a Lentivirus or a Spumavirus. Oncovirus antigens may be derived from HTLV-1, HTLV-2 or HTLV-5. Lentivirus antigens may be derived from HIV-1 or HIV-2. Retrovirus antigens may be selected from gag, pol, env, tax, tat, rex, rev, nef, vif, vpu, and vpr. HIV antigens may be selected from gag (p24gag and p55gag), env (gp160 and gp41), pol, tat, nef, rev vpu, miniproteins, (preferably p55 gag and gp140v delete). HIV antigens may be derived from one or more of the following strains: HIV_{IIIb}, HIV_{SF2}, HIV_{LAV}, HIV_{LAI}, HIV_{MN}, HIV-1_{CM235}, HIV-1_{US4}.

Reovirus: Viral antigens may be derived from a Reovirus, such as an Orthoreovirus, a Rotavirus, an Orbivirus, or a Coltivirus. Reovirus antigens may be selected from structural proteins λ1, λ2, λ3, μ1, μ2, σ1, σ2, or σ3, or nonstructural proteins σNS, μNS, or σ1s. Preferred Reovirus antigens may be derived from a Rotavirus. Rotavirus antigens may be selected from VP1, VP2, VP3, VP4 (or the cleaved product VP5 and VP8), NSP 1, VP6, NSP3, NSP2, VP7, NSP4, or NSP5. Preferred Rotavirus antigens include VP4 (or the cleaved product VP5 and VP8), and VP7.

Parvovirus: Viral antigens may be derived from a Parvovirus, such as Parvovirus B19. Parvovirus antigens may be selected from VP-1, VP-2, VP-3, NS-1 and NS-2. Preferably, the Parvovirus antigen is capsid protein VP-2.

Delta hepatitis virus (HDV): Viral antigens may be derived HDV, particularly δ-antigen from HDV (see, e.g., U.S. Pat. No. 5,378,814).

Hepatitis E virus (HEV): Viral antigens may be derived from HEV.

Hepatitis G virus (HGV): Viral antigens may be derived from HGV.

Human Herpesvirus: Viral antigens may be derived from a Human Herpesvirus, such as Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8). Human Herpesvirus antigens may be selected from immediate early proteins (α), early proteins (β), and late proteins (γ). HSV antigens may be derived from HSV-1 or HSV-2 strains. HSV antigens may be selected from glycoproteins gB, gC, gD and gH, fusion protein (gB), or immune escape proteins (gC, gE, or gI). VZV antigens may be selected from core, nucleocapsid, tegument, or envelope proteins. A live attenuated VZV vaccine is commercially available. EBV antigens may be selected from early antigen (EA) proteins, viral capsid antigen (VCA), and glycoproteins of the membrane antigen (MA). CMV antigens may be selected from capsid proteins, envelope glycoproteins (such as gB and gH), and tegument proteins

Papovaviruses: Antigens may be derived from Papovaviruses, such as Papillomaviruses and Polyomaviruses. Papillomaviruses include HPV serotypes 1, 2, 4, 5, 6, 8, 11, 13, 16, 18, 31, 33, 35, 39, 41, 42, 47, 51, 57, 58, 63 and 65. Preferably, HPV antigens are derived from serotypes 6, 11, 16 or 18. HPV antigens may be selected from capsid proteins (L1) and (L2), or E1-E7, or fusions thereof. HPV antigens are preferably formulated into virus-like particles (VLPs). Polyomyavirus viruses include BK virus and JK virus. Polyomavirus antigens may be selected from VP 1, VP2 or VP3.

Further provided are antigens, compositions, methods, and microbes included in Vaccines, 4^{th} Edition (Plotkin and Orenstein ed. 2004); Medical Microbiology 4^{th} Edition (Murray et al. ed. 2002); Virology, 3rd Edition (W. K. Joklik ed. 1988); Fundamental Virology, 2nd Edition (B. N. Fields and D. M. Knipe, eds. 1991), which are contemplated in conjunction with the compositions of the present invention.

### C. Fungal Antigens

Fungal antigens for use in the invention may be derived from one or more of the fungi set forth below.

Fungal antigens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme.

Fungal pathogens may be derived from Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Processes for producing a fungal antigens are well known in the art (see U.S. Pat. No. 6,333,164). In a preferred method a solubilized fraction extracted and separated from an insoluble fraction obtainable from fungal cells of which cell wall has been substantially removed or at least partially removed, characterized in that the process comprises the steps of: obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

### D. STD Antigens

The compositions of the invention may include one or more antigens derived from a sexually transmitted disease (STD). Such antigens may provide for prophylactis or therapy for STD's such as chlamydia, genital herpes, hepatits (such as HCV), genital warts, gonorrhoea, syphilis and/or chancroid (See, WO00/15255). Antigens may be derived from one or more viral or bacterial STD's. Viral STD antigens for use in the invention may be derived from, for example, HIV, herpes simplex virus (HSV-1 and HSV-2), human papillomavirus (HPV), and hepatitis (HCV). Bacterial STD antigens for use in the invention may be derived from, for example, Neiserria gonorrhoeae, Chlamydia trachomatis, Treponema pallidum, Haemophilus ducreyi, E. coli, and Streptococcus agalactiae. Examples of specific antigens derived from these pathogens are described above.

### E. Respiratory Antigens

The compositions of the invention may include one or more antigens derived from a pathogen which causes respiratory disease. For example, respiratory antigens may be derived from a respiratory virus such as Orthomyxoviruses (influenza), Pneumovirus (RSV), Paramyxovirus (PIV), Morbillivirus (measles), Togavirus (Rubella), VZV, and Coronavirus (SARS). Respiratory antigens may be derived from a bacteria which causes respiratory disease, such as Streptococcus pneumoniae, Pseudomonas aeruginosa, Bordetella pertussis, Mycobacterium tuberculosis, Mycoplasma pneumoniae, Chlamydia pneumoniae, Bacillus anthracis, and Moraxella catarrhalis. Examples of specific antigens derived from these pathogens are described above.

### F. Pediatric Vaccine Antigens

The compositions of the invention may include one or more antigens suitable for use in pediatric subjects. Pediatric subjects are typically less than about 3 years old, or less than about 2 years old, or less than about 1 years old. Pediatric antigens may be administered multiple times over the course of 6 months, 1, 2 or 3 years. Pediatric antigens may be derived from a virus which may target pediatric populations and/or a virus from which pediatric populations are susceptible to infection. Pediatric viral antigens include antigens derived from one or more of Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), and Varicella-zoster virus (VZV), Epstein Barr virus (EBV). Pediatric bacterial antigens include antigens derived from one or more of Streptococcus pneumoniae, Neisseria meningitides, Streptococcus pyogenes (Group A Streptococcus), Moraxella catarrhalis, Bordetella pertussis, Staphylococcus aureus, Clostridium tetani (Tetanus), Cornynebacterium diphtheriae (Diphtheria), Haemophilus influenzae B (Hib), Pseudomonas aeruginosa, Streptococcus agalactiae (Group B Streptococcus), and E. coli. Examples of specific antigens derived from these pathogens are described above.

### G. Antigens Suitable for Use in Elderly or Immunocompromised Individuals

The compositions of the invention may include one or more antigens suitable for use in elderly or immunocompromised individuals. Such individuals may need to be vaccinated more frequently, with higher doses or with adjuvanted formulations to improve their immune response to the targeted antigens. Antigens which may be targeted for use in Elderly or Immunocompromised individuals include antigens derived from one or more of the following pathogens: Neisseria meningitides, Streptococcus pneumoniae, Streptococcus pyogenes (Group A Streptococcus), Moraxella catarrhalis, Bordetella pertussis, Staphylococcus aureus, Staphylococcus epidermis, Clostridium tetani (Tetanus), Cornynebacterium diphtheriae (Diphtheria), Haemophilus influenzae B (Hib), Pseudomonas aeruginosa, Legionella pneumophila, Streptococcus agalactiae (Group B Streptococcus), Enterococcus faecalis, Helicobacter pylori, Clamydia pneumoniae, Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), Varicella-zoster virus (VZV), Epstein Barr virus (EBV), Cytomegalovirus (CMV). Examples of specific antigens derived from these pathogens are described above.

### H. Antigens Suitable for Use in Adolescent Vaccines

The compositions of the invention may include one or more antigens suitable for use in adolescent subjects. Adolescents may be in need of a boost of a previously administered pediatric antigen. Pediatric antigens which may be suitable for use in adolescents are described above. In addition, adolescents may be targeted to receive antigens derived from an STD pathogen in order to ensure protective or therapeutic immunity before the beginning of sexual activity. STD antigens which may be suitable for use in adolescents are described above.

### I. Tumor Antigens

One embodiment of the present involves a tumor antigen or cancer antigen in conjunction with the compositions of the present invention. Tumor antigens can be, for example, peptide-containing tumor antigens, such as a polypeptide tumor antigen or glycoprotein tumor antigens. A tumor antigen can also be, for example, a saccharide-containing tumor antigen, such as a glycolipid tumor antigen or a ganglioside tumor antigen. The tumor antigen can further be, for example, a polynucleotide-containing tumor antigen that expresses a polypeptide-containing tumor antigen, for instance, an RNA vector construct or a DNA vector construct, such as plasmid DNA.

Tumor antigens appropriate for the practice of the present invention encompass a wide variety of molecules, such as (a) polypeptide-containing tumor antigens, including polypeptides (which can range, for example, from 8-20 amino acids in length, although lengths outside this range are also common), lipopolypeptides and glycoproteins, (b) saccharide-containing tumor antigens, including poly-saccharides, mucins, gangliosides, glycolipids and glycoproteins, and (c) polynucleotides that express antigenic polypeptides.

The tumor antigens can be, for example, (a) full length molecules associated with cancer cells, (b) homologs and modified forms of the same, including molecules with deleted, added and/or substituted portions, and (c) fragments of the same. Tumor antigens can be provided in recombinant form. Tumor antigens include, for example, class 1-restricted antigens recognized by CD8+ lymphocytes or class II-restricted antigens recognized by CD4+ lymphocytes.

Numerous tumor antigens are known in the art, including: (a) cancer-testis antigens such as NY-ESO-1, SSX₂, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT, (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma), (e) prostate associated antigens such as PAP, PSA, PSMA, PSH-P1, PSM-P1, PSM-P2, associated with e.g., prostate cancer, (f) immunoglobulin idiotypes (associated with myeloma and B cell lymphomas, for example), and (g) other tumor antigens, such as polypeptide- and saccharide-containing antigens including (i) glycoproteins such as sialyl Tn and sialyl Le^{x} (associated with, e.g., breast and colorectal cancer) as well as various mucins; glycoproteins may be coupled to a carrier protein (e.g., MUC-1 may be coupled to KLH); (ii) lipopolypeptides (e.g., MUC-1 linked to a lipid moiety); (iii) polysaccharides (e.g., Globo H synthetic hexasaccharide), which may be coupled to a carrier proteins (e.g., to KLH), (iv) gangliosides such as GM2, GM12, GD2, GD3 (associated with, e.g., brain, lung cancer, melanoma), which also may be coupled to carrier proteins (e.g., KLH). Additional tumor antigens which are known in the art include p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68KP1, CO-029, FGF-5, Ga733 (EpCAM), HTgp-175, M344, MA-50, MG7-Ag, MOV18, NB/70K, NY—CO-1, RCAS1, SDCCAG16, TA-90 (Mac-2 binding protein\cyclophilin C-associated protein), TAAL6, TAG72, TLP, TPS, and the like. These as well as other cellular components are described for example in United States Patent Application 20020007173 and references cited therein.

Polynucleotide-containing antigens in accordance with the present invention typically comprise polynucleotides that encode polypeptide cancer antigens such as those listed above. Preferred polynucleotide-containing antigens include DNA or RNA vector constructs, such as plasmid vectors (e.g., pCMV), which are capable of expressing polypeptide cancer antigens in vivo.

Tumor antigens may be derived, for example, from mutated or altered cellular components. After alteration, the cellular components no longer perform their regulatory functions, and hence the cell may experience uncontrolled growth. Representative examples of altered cellular components include ras, p53, Rb, altered protein encoded by the Wilms' tumor gene, ubiquitin, mucin, protein encoded by the DCC, APC, and MCC genes, as well as receptors or receptor-like structures such as neu, thyroid hormone receptor, platelet derived growth factor (PDGF) receptor, insulin receptor, epidermal growth factor (EGF) receptor, and the colony stimulating factor (CSF) receptor. These as well as other cellular components are described for example in U.S. Pat. No. 5,693,522 and references cited therein.

Additionally, bacterial and viral antigens, may be used in conjunction with the compositions of the present invention for the treatment of cancer. In particular, carrier proteins, such as CRM₁₉₇, tetanus toxoid, or Salmonella typhimurium antigen can be used in conjunction/conjugation with compounds of the present invention for treatment of cancer. The cancer antigen combination therapies will show increased efficacy and bioavailability as compared with existing therapies.

Additional information on cancer or tumor antigens can be found, for example, in Moingeon P, “Cancer vaccines,” Vaccine, 2001, 19:1305-1326; Rosenberg S A, “Progress in human tumor immunology and immunotherapy,” Nature, 2001, 411:380-384; Dermine, S. et al, “Cancer Vaccines and Immunotherapy,” British Medical Bulletin, 2002, 62, 149-162; Espinoza-Delgado I., “Cancer Vaccines,” The Oncologist, 2002, 7(suppl3):20-33; Davis, I. D. et al., “Rational approaches to human cancer immunotherapy,” Journal of Leukocyte Biology, 2003, 23: 3-29; Van den Eynde B, et al., “New tumor antigens recognized by T cells,” Curr. Opin. Immunol., 1995, 7:674-81; Rosenberg S A, “Cancer vaccines based on the identification of genes encoding cancer regression antigens, Immunol. Today, 1997, 18:175-82; Offringa R et al., “Design and evaluation of antigen-specific vaccination strategies against cancer,” Current Opin. Immunol., 2000, 2:576-582; Rosenberg S A, “A new era for cancer immunotherapy based on the genes that encode cancer antigens,” Immunity, 1999, 10:281-7; Sahin U et al., “Serological identification of human tumor antigens,” Curr. Opin. Immunol., 1997, 9:709-16; Old L J et al., “New paths in human cancer serology,” J. Exp. Med., 1998, 187:1163-7; Chaux P, et al., “Identification of MAGE-3 epitopes presented by HLA-DR molecules to CD4(+) T lymphocytes,” J. Exp. Med., 1999, 189:767-78; Gold P, et al., “Specific carcinoembryonic antigens of the human digestive system,” J. Exp. Med., 1965, 122:467-8; Livingston P O, et al., Carbohydrate vaccines that induce antibodies against cancer: Rationale,” Cancer Immunol. Immunother., 1997, 45:1-6; Livingston P O, et al., Carbohydrate vaccines that induce antibodies against cancer: Previous experience and future plans,” Cancer Immunol. Immunother., 1997, 45:10-9; Taylor-Papadimitriou J, “Biology, biochemistry and immunology of carcinoma-associated mucins,” Immunol. Today, 1997, 18:105-7; Zhao X-J et al., “GD2 oligosaccharide: target for cytotoxic T lymphocytes,” J. Exp. Med., 1995, 182:67-74; Theobald M, et al., “Targeting p53 as a general tumor antigen,” Proc. Natl. Acad. Sci. USA, 1995, 92:11993-7; Gaudernack G, “T cell responses against mutant ras: a basis for novel cancer vaccines,” Immunotechnology, 1996, 2:3-9; WO 91/02062; U.S. Pat. No. 6,015,567; WO 01/08636; WO 96/30514; U.S. Pat. No. 5,846,538; and U.S. Pat. No. 5,869,445.

### J. Antigen Formulations

In other aspects of the invention, methods of producing microparticles having adsorbed antigens are provided. The methods comprise: (a) providing an emulsion by dispersing a mixture comprising (i) water, (ii) a detergent, (iii) an organic solvent, and (iv) a biodegradable polymer selected from the group consisting of a poly(α-hydroxy acid), a polyhydroxy butyric acid, a polycaprolactone, a polyorthoester, a polyanhydride, and a polycyanoacrylate. The polymer is typically present in the mixture at a concentration of about 1% to about 30% relative to the organic solvent, while the detergent is typically present in the mixture at a weight-to-weight detergent-to-polymer ratio of from about 0.00001:1 to about 0.1:1 (more typically about 0.0001:1 to about 0.1:1, about 0.001:1 to about 0.1:1, or about 0.005:1 to about 0.1:1); (b) removing the organic solvent from the emulsion; and (c) adsorbing an antigen on the surface of the microparticles. In certain embodiments, the biodegradable polymer is present at a concentration of about 3% to about 10% relative to the organic solvent.

Microparticles for use herein will be formed from materials that are sterilizable, non-toxic and biodegradable. Such materials include, without limitation, poly(α-hydroxy acid), polyhydroxybutyric acid, polycaprolactone, polyorthoester, polyanhydride, PACA, and polycyanoacrylate. Preferably, microparticles for use with the present invention are derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the coadministered macromolecule. These parameters are discussed more fully below.

Further antigens may also include an outer membrane vesicle (OMV) preparation.

Additional formulation methods and antigens (especially tumor antigens) are provided in U.S. patent Ser. No. 09/581,772.

### K. Antigen References

The following references include antigens useful in conjunction with the compositions of the present invention:

Antigen references are listed below:
1. International patent application WO 99/24578
2. International patent application WO 99/36544.
3. International patent application WO 99/57280.
4. International patent application WO 00/22430.
5. Tettelin et al. (2000) Science 287:1809-1815.
6. International patent application WO 96/29412.
7. Pizza et al. (2000) Science 287:1816-1820.
8. PCT WO 01/52885.
9. Bjune et al. (1991) Lancet 338(8775).
10. Fuskasawa et al. (1999) Vaccine 17:2951-2958.
11. Rosenqist et al. (1998) Dev. Biol. Strand 92:323-333.
12. Constantino et al. (1992) Vaccine 10:691-698.
13. Constantino et al. (1999) Vaccine 17:1251-1263.
14. Watson (2000) Pediatr Infect Dis J 19:331-332.
15. Rubin (20000) Pediatr Clin North Am 47:269-285,v.
16. Jedrzejas (2001) Microbiol Mol Biol Rev 65:187-207.
17. International patent application filed on 3 Jul. 2001 claiming priority from GB-0016363.4; WO 02/02606; PCT IB/01/00166.
18. Kalman et al. (1999) Nature Genetics 21:385-389.
19. Read et al. (2000) Nucleic Acids Res 28:1397-406.
20. Shirai et al. (2000) J. Infect. Dis 181(Suppl 3):S524-S527.
21. International patent application WO 99/27105.
22. International patent application WO 00/27994.
23. International patent application WO 00/37494.
24. International patent application WO 99/28475.
25. Bell (2000) Pediatr Infect Dis J 19:1187-1188.
26. Iwarson (11995) APMIS103:321-326.
27. Gerlich et al. (1990) Vaccine 8 Suppl:S63-68 & 79-80.
28. Hsu et al. (1999) Clin Liver Dis 3:901-915.
29. Gastofsson et al. (1996) N. Engl. J. Med. 334-:349-355.
30. Rappuoli et al. (1991) TIBTECH 9:232-238.
31. Vaccines (1988) eds. Plotkin & Mortimer. ISBN 0-7216-1946-0.
32. Del Guidice et al. (1998) Molecular Aspects of Medicine 19:1-70.
33. International patent application WO 93/018150.
34. International patent application WO 99/53310.
35. International patent application WO 98/04702.
36. Ross et al. (2001) Vaccine 19:135-142.
37. Sutter et al. (2000) Pediatr Clin North Am 47:287-308.
38. Zimmerman & Spann (1999) Am Fan Physician 59:113-118, 125-126.
39. Dreensen (1997) Vaccine 15 Suppl″S2-6.
40. MMWR Morb Mortal Wkly rep 1998 Jan. 16:47(1):12, 9.
41. McMichael (2000) Vaccine 19 Suppl 1: S101-107.
42. Schuchat (1999) Lancer 353(9146):51-6.
43. GB patent applications 0026333.5, 0028727.6 & 0105640.7.
44. Dale (11999) Infect Disclin North Am 13:227-43, viii.
45. Ferretti et al. (2001) PNAS USA 98: 4658-4663.
46. Kuroda et al. (2001) Lancet 357(9264):1225-1240; see also pages 1218-1219.
47. Ramsay et al. (2001) Lancet 357(9251):195-196.
48. Lindberg (1999) Vaccine 17 Suppl 2:S28-36.
49. Buttery & Moxon (2000) J R Coil Physicians Long 34:163-168.
50. Ahmad & Chapnick (11999) Infect Dis Clin North Am 13:113-133, vii.
51. Goldblatt (1998) J. Med. Microbiol. 47:663-567.
52. European patent 0 477 508.
53. U.S. Pat. No. 5,306,492.
54. International patent application WO 98/42721.
55. Conjugate Vaccines (eds. Cruse et al.) ISBN 3805549326, particularly vol. 10:48-114.
56. Hermanson (1996) Bioconjugate Techniques ISBN: 012323368 & 012342335X.
57. European patent application 0372501.
58. European patent application 0378881.
59. European patent application 0427347.
60. International patent application WO 93/17712.
61. International patent application WO 98/58668.
62. European patent application 0471177.
63. International patent application WO 00/56360.
64. International patent application WO 00/67161.

Pharmaceutical compositions that include the compounds described herein may include additives such as excipients. Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-α-cyclodextrin, polyvinylpyrrolidinone, low melting waxes, ion exchange resins, and the like, as well as combinations of any two or more of these. Other suitable pharmaceutically acceptable excipients are described in “Remington's Pharmaceutical Sciences,” Mack Pub. Co., New Jersey (1991), which is hereby incorporated herein by reference in its entirety and for all purposes as if fully set forth herein.

Pharmaceutical compositions that include the compounds of the invention may be in any form suitable for the intended method of administration, including, for example, as a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more of these. The liquid carrier may include other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, but are not limited to, soybean oil, coconut oil, olive oil, safflower oil, cottonseed oil, and the like. For parenteral administration, the carrier may be an oily ester such as ethyl oleate, isopropyl myristate, and the like. Compositions of the present invention may also be in the form of microparticles, microcapsules, and the like, as well as combinations of any two or more of these.

The compounds and combinations of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form may include, in addition to a compound of the present invention, stabilizers, preservatives, excipients, and the like. Preferred lipids include phospholipids and phosphatidyl cholines (lecithins), both natural and synthetic. Methods of forming liposomes are known in the art. See, for example, Prescott, Ed., Methods in Cell Biology, Volume XIV, Academic Press, New York, N.W., p. 33 et seq (1976).

Controlled release delivery systems may also be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation spray, rectally, or topically in dosage unit formulations that include conventional nontoxic pharmaceutically acceptable carriers, adjuvants, and vehicles as desired. For example, suitable modes of administration include oral, subcutaneous, transdermal, transmucosal, iontophoretic, intravenous, intramuscular, intraperitoneal, intranasal, subdermal, rectal, and the like. Topical administration may also include the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at ordinary temperatures but liquid at the rectal temperature and will, therefore, melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also include, as is normal practice, additional substances other than inert diluents, e.g., lubricating agents such as magnesium stearate. In the case of capsules, tablets, and pills, the dosage forms may also include buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compositions of the invention can further be combined with antigens as above and or adjuvants and other immune stimulators as described below.

### Adjuvants:

Vaccine compositions contemplated to be within the scope of the present invention may include (an) additional adjuvant(s) and or other immune stimulator compound.

### Adjuvants

Vaccines or immunogenic compositions of the invention may be administered in conjunction with other immunoregulatory agents. In particular, compositions will usually include an adjuvant. Adjuvants for use with the invention include, but are not limited to, one or more of the following set forth below:

### Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design . . . (1995) eds. Powell & Newman. ISBN: 030644867X. Plenum.), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO00/23105).

Aluminum salts may be included in vaccines of the invention such that the dose of Al³⁺ is between 0.2 and 1.0 mg per dose.

In one embodiment the aluminum based adjuvant for use in the present invention is alum (aluminum potassium sulfate (AlK(SO₄)₂)), or an alum derivative, such as that formed in-situ by mixing an antigen in phosphate buffer with alum, followed by titration and precipitation with a base such as ammonium hydroxide or sodium hydroxide.

Another aluminum-based adjuvant for use in vaccine formulations of the present invention is aluminum hydroxide adjuvant (Al(OH)₃) or crystalline aluminum oxyhydroxide (AlOOH), which is an excellent adsorbent, having a surface area of approximately 500 m²/g. Alternatively, aluminum phosphate adjuvant (AlPO₄) or aluminum hydroxyphosphate, which contains phosphate groups in place of some or all of the hydroxyl groups of aluminum hydroxide adjuvant is provided. Preferred aluminum phosphate adjuvants provided herein are amorphous and soluble in acidic, basic and neutral media.

In another embodiment the adjuvant of the invention comprises both aluminum phosphate and aluminum hydroxide. In a more particular embodiment thereof, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. More particular still, aluminum salts in the vaccine are present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (iep=4) adsorbs lysozyme, but not albumin at pH 7.4. Should albumin be the target, aluminum hydroxide adjuvant would be selected (iep 11.4). Alternatively, pretreatment of aluminum hydroxide with phosphate lowers its isoelectric point, making it a preferred adjuvant for more basic antigens.

### Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See WO90/14837. See also, Podda, “The adjuvanted influenza vaccines with novel adjuvants: experience with the MF59-adjuvanted vaccine”, Vaccine (2001) 19: 2673-2680; Frey et al., “Comparison of the safety, tolerability, and immunogenicity of a MF59-adjuvanted influenza vaccine and a non-adjuvanted influenza vaccine in non-elderly adults”, Vaccine (2003) 21:4234-4237. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphosphoryloxy)-ethylamine (MTP-PE), for example, the submicron oil-in-water emulsion known as “MF59” (International Publication No. WO90/14837; U.S. Pat. Nos. 6,299,884 and 6,451,325, and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) may also be used as adjuvants in the invention.

Specific oil-in-water emulsion adjuvants useful with the invention include, but are not limited to:
(1) A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [WO90/14837.—Podda & Del Giudice (2003) Expert Rev Vaccines 2:197-203. Podda (2001) Vaccine 19: 2673-2680.], as described in more detail in Chapter 10 of ref. Vaccine Design: The Subunit and Adjuvant Approach (eds. Powell & Newman) Plenum Press 1995 (ISBN 0-306-44867-X). and chapter 12 of ref. Vaccine Adjuvants: Preparation Methods and Research Protocols (Volume 42 of Methods in Molecular Medicine series). ISBN: 1-59259-083-7. Ed. O'Hagan. The MF59 emulsion advantageously includes citrate ions e.g. 10 mM sodium citrate buffer.
(2) An emulsion of squalene, a tocopherol, and Tween 80. The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is preferably ≦1 as this provides a more stable emulsion. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.
(3) An emulsion of squalene, a tocopherol, and a Triton detergent (e.g. Triton X-100).
(4) An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [Allison & Byars (1992) Res Immunol 143:519-25] (0.05-1% Thr-MDP, 5% squalane, 2.5% Pluronic L121 and 0.2% polysorbate 80). It can also be used without the Thr-MDP, as in the “AF” adjuvant [Hariharan et al. (1995) Cancer Res 55:3486-9] (5% squalane, 1.25% Pluronic L121 and 0.2% polysorbate 80). Microfluidisation is preferred.

The emulsions are preferably mixed with additional agents (such as an antigen) extemporaneously, at the time of delivery. Thus the adjuvant and antigen are typically kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g. between 5:1 and 1:5) but is generally about 1:1.

Where a composition includes a tocopherol, any of the α, β, γ, δ, ε or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in vaccines for use in elderly patients (e.g. aged 60 years or older) because vitamin E has been reported to have a positive effect on the immune response in this patient group [Han et al (2005) Impact of Vitamin E on Immune Function and Infectious Diseases in the Aged at Nutrition, Immune functions and Health EuroConference, Paris, 9-10 Jun. 2005]. They also have antioxidant properties that may help to stabilize the emulsions [U.S. Pat. No. 6,630,161]. A preferred α-tocopherol is DL-α-tocopherol, and the preferred salt of this tocopherol is the succinate. The succinate salt has been found to cooperate with TNF-related ligands in vivo. Moreover, α-tocopherol succinate is known to be compatible with influenza vaccines and to be a useful preservative as an alternative to mercurial compounds

### Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO96/11711 and WO96/33739. Optionally, the ISCOMS may be devoid of (an) additional detergent(s). See WO00/07621.

A review of the development of saponin based adjuvants can be found in Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

### Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO03/024480, WO03/024481, and Niikura et al., “Chimeric Recombinant Hepatitis E Virus-Like Particles as an Oral Vaccine Vehicle Presenting Foreign Epitopes”, Virology (2002) 293:273-280; Lenz et al., “Papillomarivurs-Like Particles Induce Acute Activation of Dendritic Cells”, Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338; and Gerber et al., “Human Papillomavirus Virus-Like Particles Are Efficient Oral Immunogens when Coadministered with Escherichia coli Heat-Labile Entertoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B10-B16. Immunopotentiating reconstituted influenza virosomes (IRIV) are used as the subunit antigen delivery system in the intranasal trivalent INFLEXAL™ product {Mischler & Metcalfe (2002) Vaccine 20 Suppl 5:B17-23} and the INFLUVAC PLUS™ product.

### Bacterial or Microbial Derivatives

Adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

3dMPL has been prepared from a heptoseless mutant of Salmonella minnesota. It activates cells of the monocyte/macrophage lineage and stimulates release of several cytokines, including IL-1, IL-12, TNF-α and GM-CSF (see also ref. Thompson et al. (2005) J Leukoc Biol 78: ‘The low-toxicity versions of LPS, MPL® adjuvant and RC529, are efficient adjuvants for CD4+ T cells’.). Preparation of 3dMPL was originally described in reference UK patent application GB-A-2220211.

3dMPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1′}. The group attached to carbon 2′ has formula —NH—CO—CH₂—CR²R^{2′}. The group attached to carbon 3′ has formula —O—CO—CH₂—CR³R^{3′}. A representative structure is:

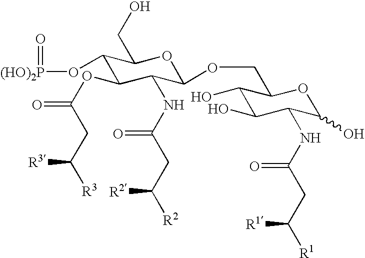

Groups R¹, R² and R³ are each independently —(CH₂)ₙ—CH₃. The value of n is preferably between 8 and 16, more preferably between 9 and 12, and is most preferably 10. Groups R^{1′}, R^{2′} and R^{3′} can each independently be: (a) —H; (b) —OH; or (c) —O—CO—R⁴, where R⁴ is either —H or —(CH₂)ₘ—CH₃, wherein the value of m is preferably between 8 and 16, and is more preferably 10, 12 or 14. At the 2 position, m is preferably 14. At the 2′ position, m is preferably 10. At the 3′ position, m is preferably 12. Groups R^{1′}, R^{2′} and R^{3′} are thus preferably —O-acyl groups from dodecanoic acid, tetradecanoic acid or hexadecanoic acid.

When all of R^{1′}, R^{2′} and R^{3′} are —H then the 3dMPL has only 3 acyl chains (one on each of positions 2, 2′ and 3′). When only two of R^{1′}, R^{2′} and R^{3′} are —H then the 3dMPL can have 4 acyl chains. When only one of R^{1′}, R^{2′} and R^{3′} is —H then the 3dMPL can have 5 acyl chains. When none of R^{1′}, R^{2′} and R^{3′} is —H then the 3dMPL can have 6 acyl chains. The 3dMPL adjuvant used according to the invention can be a mixture of these forms, with from 3 to 6 acyl chains, but it is preferred to include 3dMPL with 6 acyl chains in the mixture, and in particular to ensure that the hexaacyl chain form makes up at least 10% by weight of the total 3dMPL e.g. ≧20%, ≧30%, ≧40%, ≧50% or more. 3dMPL with 6 acyl chains has been found to be the most adjuvant-active form.

Thus the most preferred form of 3dMPL for inclusion in compositions of the invention is:

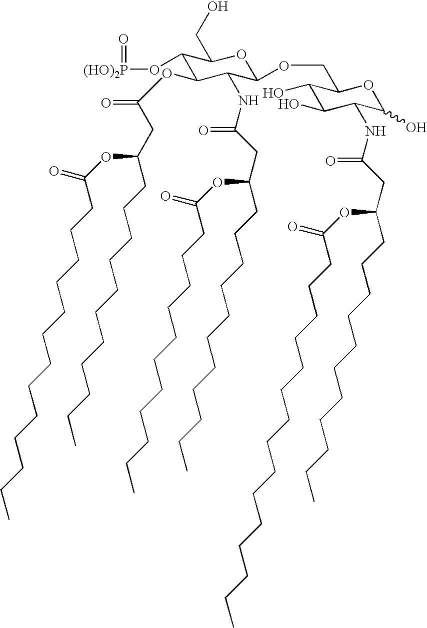

Where 3dMPL is used in the form of a mixture then references to amounts or concentrations of 3dMPL in compositions of the invention refer to the combined 3dMPL species in the mixture.

In aqueous conditions, 3dMPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3dMPL) are preferred for use according to the invention because of their superior activity [WO 94/21292]. Preferred particles have a mean diameter less than 220 nm, more preferably less than 200 nm or less than 150 nm or less than 120 nm, and can even have a mean diameter less than 100 nm. In most cases, however, the mean diameter will not be lower than 50 nm. These particles are small enough to be suitable for filter sterilization. Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≧60%, ≧70%, ≧80%, ≧90%, or more) of the particles will have a diameter within the range x±25%.

3dMPL can advantageously be used in combination with an oil-in-water emulsion. Substantially all of the 3dMPL may be located in the aqueous phase of the emulsion. The 3dMPL can be used on its own, or in combination with one or more further compounds. For example, it is known to use 3dMPL in combination with the QS21 saponin [WO94/00153.] (including in an oil-in-water emulsion [WO95/17210]), with an immunostimulatory oligonucleotide, with both QS21 and an immunostimulatory oligonucleotide, with aluminum phosphate [WO96/26741], with aluminum hydroxide [WO93/19780], or with both aluminum phosphate and aluminum hydroxide.

### Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.

### Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068; Krieg, “From A to Z on CpG”, TRENDS in Immunology (2002) 23(2): 64-65 and WO01/95935. Preferably, the CpG is a CpG-A ODN.

Examples of CpG nucleotides include the following sequences, which may contain phosphorothioate modified internucleotide linkages:

TCC ATG ACG TTC CTG ACG TT (CpG 1826);
TCT CCC AGC GTG CGC CAT (CpG 1758);
ACC GAT GAC GTC GCC GGT GAC GGC ACC ACG;
TCG TCG TTT TGT CGT TTT GTC GTT (CpG 2006);
and
TCC ATG ACG TTC CTG ATG CT (CpG 1668).

### See WO 05/25614.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31 (part 3):664-658; Bhagat et al., “CpG penta- and hexadeoxyribonucleotides as potent immunomodulatory agents” BBRC (2003) 300:853-861 and WO03/035836.

### ADP-Ribosylating Toxins and Detoxified Derivatives Thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon, et al., “The LTR72 Mutant of Heat-Labile Enterotoxin of Escherichia coli Enahnces the Ability of Peptide Antigens to Elicit CD4+ T Cells and Secrete Gamma Interferon after Coapplication onto Bare Skin”, Infection and Immunity (2002) 70(6):3012-3019; Pizza, et al., “Mucosal vaccines: non toxic derivatives of LT and CT as mucosal adjuvants”, Vaccine (2001) 19:2534-2541; Pizza, et al., “LTK63 and LTR72, two mucosal adjuvants ready for clinical trials” Int. J. Med. Microbiol. (2000) 290(4-5):455-461; Scharton-Kersten et al., “Transcutaneous Immunization with Bacterial ADP-Ribosylating Exotoxins, Subunits and Unrelated Adjuvants”, Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216; Peppoloni et al., “Mutants of the Escherichia coli heat-labile enterotoxin as safe and strong adjuvants for intranasal delivery of vaccines”, Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol. (1995) 15(6):1165-1167.

### Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the invention. E.g. WO99/27960.

### Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### Liposomes

Examples of liposome formulations suitable for use as adjuvants are described in U.S. Pat. No. 6,090,406, U.S. Pat. No. 5,916,588, and EP 0 626 169. Polyoxyethylene ether and Polyoxyethylene Ester Formulations

Adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109-115 and Payne et al., “Protein Release from Polyphosphazene Matrices”, Adv. Drug. Delivery Review (1998) 31(3):185-196.

### Muramyl Peptides

Examples of muramyl peptides suitable for use as adjuvants in the invention include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

### Small Molecule Immunopontentiators (SMIPS)

### Imidazoquinoline Compounds

Examples of imidazoquinoline compounds suitable for use adjuvants in the invention include Imiquimod and its analogues, described further in Stanley, “Imiquimod and the imidazoquinolines: mechanism of action and therapeutic potential” Clin Exp Dermatol (2002) 27(7):571-577; Jones, “Resiquimod 3M”, Curr Opin Investig Drugs (2003) 4(2):214-218; Wu et al. (2004) Antiviral Res. 64(2):79-83 Vasilakos et al. (2000) Cell Immunol. 204(1):64-74 U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.

Preferred SMIPs include:
N2-methyl-1-(2-methylpropyl)-1H-imidazo[4,5-c]quinoline-2,4-diamine;
N2,N2-dimethyl-1-(2-methylpropyl)-1H-imidazo[4,5-c]quinoline-2,4-diamine;
N2-ethyl-N2-methyl-1-(2-methylpropyl)-1H-imidazo[4,5-c]quinoline-2,4-diamine;
N2-methyl-1-(2-methylpropyl)-N2-propyl-1H-imidazo[4,5-c]quinoline-2,4-diamine;
1-(2-methylpropyl)-N2-propyl-1H-imidazo[4,5-c]quinoline-2,4-diamine;
N2-butyl-1-(2-methylpropyl)-1H-imidazo[4,5-c]quinoline-2,4-diamine;
N2-butyl-N2-methyl-1-(2-methylpropyl)-1H-imidazo[4,5-c]quinoline-2,4-diamine;
N2-methyl-1-(2-methylpropyl)-N2-pentyl-1H-imidazo[4,5-c]quinoline-2,4-diamine;
N2-methyl-1-(2-methylpropyl)-N2-prop-2-enyl-1H-imidazo[4,5-c]quinoline-2,4-diamine;
1-(2-methylpropyl)-2-[(phenylmethyl)thio]-1H-imidazo[4,5-c]quinolin-4-amine;
1-(2-methylpropyl)-2-(propylthio)-1H-imidazo[4,5-c]quinolin-4-amine;
2-[[4-amino-1-(2-methylpropyl)-1H-imidazo[4,5-c]quinolin-2-yl] (methyl)amino]ethanol;
2-[[4-amino-1-(2-methylpropyl)-1H-imidazo[4,5-c]quinolin-2-yl](methyl)amino]ethyl acetate;
4-amino-1-(2-methylpropyl)-1,3-dihydro-2H-imidazo[4,5-c]quinolin-2-one;
N2-butyl-1-(2-methylpropyl)-N4,N4-bis(phenylmethyl)-1H-imidazo[4,5-c]quinoline-2,4-diamine;
N2-butyl-N2-methyl-1-(2-methylpropyl)-N4,N4-bis(phenylmethyl)-1H-imidazo[4,5-c]quinoline-2,4-diamine;
N2-methyl-1-(2-methylpropyl)-N4,N4-bis(phenylmethyl)-1H-imidazo[4,5-c]quinoline-2,4-diamine;
N2,N2-dimethyl-1-(2-methylpropyl)-N4,N4-bis(phenylmethyl)-1H-imidazo[4,5-c]quinoline-2,4-diamine;
1-{4-amino-2-[methyl(propyl)amino]-1H-imidazo[4,5-c]quinolin-1-yl}-2-methylpropan-2-ol;
1-[4-amino-2-(propylamino)-1H-imidazo[4,5-c]quinolin-1-yl]-2-methylpropan-2-ol;
N4,N4-dibenzyl-1-(2-methoxy-2-methylpropyl)-N2-propyl-1H-imidazo[4,5-c]quinoline-2,4-diamine.

### Nucleoside Analogs.

A nucleoside analog, such as: (a) Isatorabine (ANA-245; 7-thia-8-oxoguanosine):

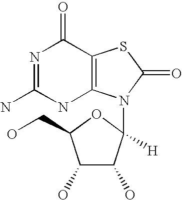

and prodrugs thereof, (b) ANA975; (c) ANA-025-1; (d) ANA380; (e) the compounds disclosed in references U.S. Pat. No. 6,924,271 to US2005/0070556 U.S. Pat. No. 5,658,731; (f) a compound having the formula:

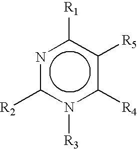

wherein:
R₁ and R₂ are each independently H, halo, —NRₐR_{b}, —OH, C₁₋₆ alkoxy, substituted C₁₋₆ alkoxy, heterocyclyl, substituted heterocyclyl, C₆₋₁₀ aryl, substituted C₆₋₁₀ aryl, C₁₋₆ alkyl, or substituted C₁₋₆ alkyl;
R₃ is absent, H, C₁₋₆ alkyl, substituted C₁₋₆ alkyl, C₆₋₁₀ aryl, substituted C₆₋₁₀ aryl, heterocyclyl, or substituted heterocyclyl;
R₄ and R₅ are each independently H, halo, heterocyclyl, substituted heterocyclyl, —C(O)—R_{d}, C₁₋₆ alkyl, substituted C₁₋₆ alkyl, or bound together to form a 5 membered ring as in R₄₋₅:

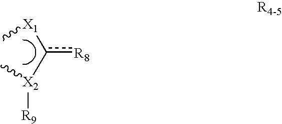

the binding being achieved at the bonds indicated by a [symbol]
X₁ and X₂ are each independently N, C, O, or S;
R₈ is H, halo, —OH, C₁₋₆ alkyl, C₂₋₆ alkenyl, C₂₋₆ alkynyl, —OH, —NRₐR_{b}, —(CH₂)ₙ—O—R_{c}, —O—(C₁₋₆ alkyl), —S(O)ₚRₑ, or —C(O)—R_{d};
R₉ is H, C₁₋₆ alkyl, substituted C₁₋₆ alkyl, heterocyclyl, substituted heterocyclyl or R₉ₐ, wherein R₉ₐ is:

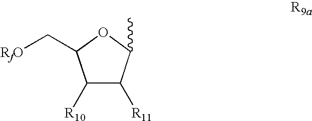

the binding being achieved at the bond indicated by a [symbol]
R₁₀ and R₁₁ are each independently H, halo, C₁₋₆ alkoxy, substituted C₁₋₆ alkoxy, —NRₐR_{b}, or —OH;
each Rₐ and R_{b} is independently H, C₁₋₆ alkyl, substituted C₁₋₆ alkyl, —C(O)R_{d}, C₆₋₁₀ aryl;
each Rₑ is independently H, phosphate, diphosphate, triphosphate, C₁₋₆ alkyl, or substituted C₁₋₆ alkyl;
each R_{d} is independently H, halo, C₁₋₆ alkyl, substituted C₁₋₆ alkyl, C₁₋₆ alkoxy, substituted C₁₋₆ alkoxy, —NH₂, —NH(C₁₋₆ alkyl), —NH(substituted C₁₋₆ alkyl), —N(C₁₋₆ alkyl)₂, —N(substituted C₁₋₆ alkyl)₂, C₆₋₁₀ aryl, or heterocyclyl;
each Rₑ is independently H, C₁₋₆ alkyl, substituted C₁₋₆ alkyl, C₆₋₁₀ aryl, substituted C₆₋₁₀ aryl, heterocyclyl, or substituted heterocyclyl;
each R_{f} is independently H, C₁₋₆ alkyl, substituted C₁₋₆ alkyl, —C(O)R_{d}, phosphate, diphosphate, or triphosphate;
each n is independently 0, 1, 2, or 3;
each p is independently 0, 1, or 2; or
or (g) a pharmaceutically acceptable salt of any of (a) to (f), a tautomer of any of (a) to (f), or a pharmaceutically acceptable salt of the tautomer;
Loxoribine (7-allyl-8-oxoguanosine) [U.S. Pat. No. 5,011,828].

### Thiosemicarbazone Compounds.

Examples of thiosemicarbazone compounds, as well as methods of formulating, manufacturing, and screening for compounds all suitable for use as adjuvants in the invention include those described in WO04/60308. The thiosemicarbazones are particularly effective in the stimulation of human peripheral blood mononuclear cells for the production of cytokines, such as TNF-α.

### Tryptanthrin Compounds.

Examples of tryptanthrin compounds, as well as methods of formulating, manufacturing, and screening for compounds all suitable for use as adjuvants in the invention include those described in WO04/64759. The tryptanthrin compounds are particularly effective in the stimulation of human peripheral blood mononuclear cells for the production of cytokines, such as TNF-α.

### Additional SMIPs

(i) Compounds disclosed in reference WO2004/87153, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds [U.S. Pat. No. 6,605,617, WO02/18383], Hydrapthalamide compounds, Benzophenone compounds, Isoxazole compounds, Sterol compounds, Quinazilinone compounds, Pyrrole compounds [WO2004/018455], Anthraquinone compounds, Quinoxaline compounds, Triazine compounds, Pyrazalopyrimidine compounds, and Benzazole compounds [WO03/082272].

(ii) Methyl inosine 5′-monophosphate (“MIMP”) [Signorelli & Hadden (2003) Int Immunopharmacol 3(8):1177-86.].

(iii) A polyhydroxlated pyrrolizidine compound [WO2004/064715], such as one having formula:

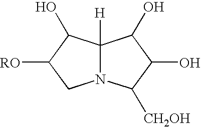

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.

(iv) A gamma inulin [Cooper (1995) Pharm Biotechnol 6:559-80] or derivative thereof, such as algammulin.

### Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

Aluminum salts and MF59 are preferred adjuvants for use with injectable i vaccines. Bacterial toxins and bioadhesives are preferred adjuvants for use with mucosally-delivered vaccines, such as nasal vaccines.

### TLR Modulators/Agonists

By “TLR agonist” it is meant a component which is capable of causing a signalling response through a TLR signalling pathway, either as a direct ligand or indirectly through generation of endogenous or exogenous ligand (Sabroe et al, Jl 2003 p 1630-5). TLR agonists of the present invention, include agonists of the following:

(1) TLR1: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)—Cys-(S)-Ser-(S) Lys(4)-OH, trihydrochloride (Pam3Cys) LP which mimics the acetylated amino terminus of a bacterial lipoprotein and OspA LP from Borrelia burgdorfei);

(2) TLR2: one or more of a bacterial lipopeptide from M tuberculosis, B burgdorferi. T pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast;

(3) TLR3: double stranded RNA, or polyinosinic-polycytidylic acid (Poly IC), a molecular nucleic acid pattern associated with viral infection;

(4) TLR4: one or more of a lipopolysaccharide (LPS) from gram-negative bacteria, or fragments thereof, heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2. In one embodiment the TLR agonist is HSP 60, 70 or 90. In an alternative embodiment, the TLR agonist capable of causing a signalling response through TLR-4 is a non-toxic derivative of LPS. Monophosphoryl lipid A (MPL) and 3D-MPL as described above, is one such non-toxic derivative. Further adjuvants and TLR4 modulators include lipids linked to a phosphate-containing acyclic backbone, such as the TLR4 antagonist E5564 [Wong et al. (2003) J Clin Pharmacol 43(7):735-42, US2005/0215517]:

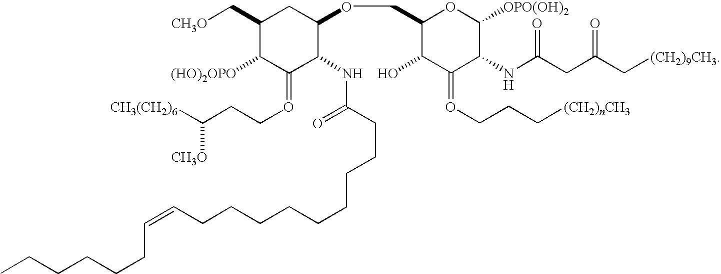

(5) TLR5: including bacterial flagellin;

(6) TLR6: including mycobacterial lipoprotein, di-acylated LP, and phenol-soluble modulin. Further TLR6 agonists are I described in WO2003043572;

(7) TLR7: including loxoribine, a guanosine analogue at positions N7 and C8, isatoribine, ANA-971, ANA-975, or an imidazoquinoline compound, or derivative thereof. In one embodiment, the TLR agonist is imiquimod or resiquimod. Further TLR7 agonists are described in WO02085905;

(8) TLR8: an imidazoquinoline molecule, for example resiquimod (R848); resiquimod is also capable of recognition by TLR-7. Other TLR-8 agonists which may be used include those described in WO2004071459; and/or

(9) TLR9: In one embodiment, I the TLR agonist capable of causing a signalling response through TLR-9 is HSP90 or a DNA containing unmethylated CpG nucleotide, in particular sequence contexts described above with CpG motifs.

Preferred TLR modulators are agonists of TLR7 (e.g. imidazoquinolines) and/or TLR9 (e.g. CpG oligonucleotides).

### Phospho-Containing Lipids

Compounds disclosed in reference PCT/JS2005/022769.

Phosphatidylcholine derivatives and phosphorylcholine containing molecules.

A compound of formula I, II or III, or a salt thereof:

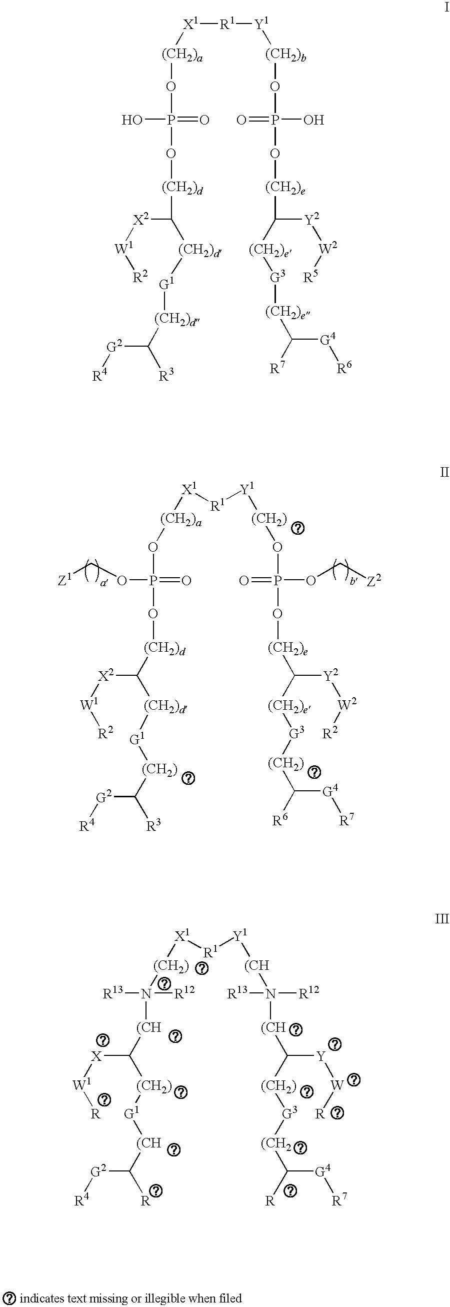

as defined in reference WO03/011223, such as ‘ER 803058’, ‘ER 803732’, ‘ER 804053’, ‘ER 804058’, ‘ER 804059’, ‘ER 804442’, ‘ER 804680’, ‘ER 804764’, ER 803022 or ‘ER 804057’ e.g.:

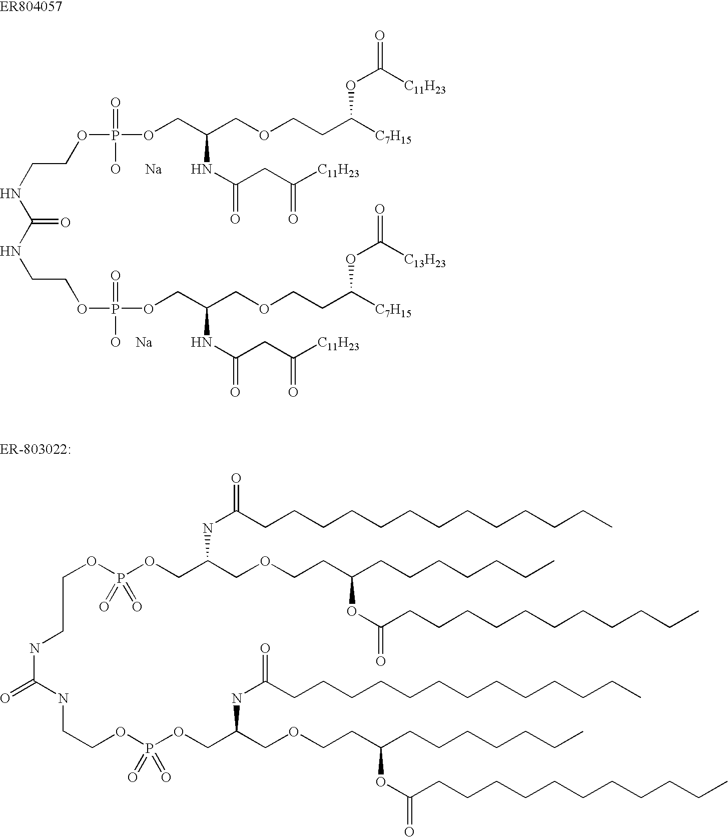

An aminoalkyl glucosaminide phosphate derivative, such as RC-529 [Johnson et al. (1999) Bioorg Med Chem Lett 9:2273-2278, Evans et al. (2003) Expert Rev Vaccines 2:219-229].

The invention may also comprise combinations of aspects of one or more of the adjuvants identified above. For example, the following adjuvant compositions may be used in the invention:

(1) a saponin and an oil-in-water emulsion (WO99/11241);

(2) a saponin (e.g., QS21)+a non-toxic LPS derivative (e.g. 3dMPL) (see WO94/00153);

(3) a saponin (e.g., QS21)+a non-toxic LPS derivative (e.g. 3dMPL)+a cholesterol;

(4) a saponin (e.g. QS21)+3dMPL+IL-12 (optionally+a sterol) (WO98/57659);

(5) combinations of 3dMPL with, for example, QS21 and/or oil-in-water emulsions (See European patent applications 0835318, 0735898 and 0761231);

(6) SAF, containing 10% Squalane, 0.4% Tween 80, 5% pluronic-block polymer L121, and thr-MDP, either microfluidized into a submicron emulsion or vortexed to generate a larger particle size emulsion.

(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and

(8) one or more mineral salts (such as an aluminum salt)+a non-toxic derivative of LPS (such as 3dPML).

(9) (9) one or more mineral salts (such as an aluminum salt)+an immunostimulatory oligonucleotide (such as a nucleotide sequence including a CpG motif).

The adjuvants described herein can be added to the composition at various stages during their production. For example, the adjuvant may be within or surround an antigen composition, and this mixture can then be/added to an oil-in-water emulsion. As an alternative, the antigen and/adjuvant may be within an oil-in-water emulsion, in which case the agent can either be added to the emulsion components before emulsification, or it can be added to the emulsion after emulsification. Similarly, the agent may be coacervated within the emulsion droplets. The location and distribution of the adjuvant within the final composition will depend on its hydrophilic/lipophilic properties e.g. the agent can be located in the aqueous phase, in the oil phase, and/or at the oil-water interface.

Further, the adjuvant described herein can be conjugated to a separate agent, such as an antigen (e.g. CRM197) or directly to any amenable composition of the present invention. A general review of conjugation techniques for small molecules is provided in Thompson et al. (2003) Methods in Molecular Medicine 94:255-266. Preferred conjugation methods involve directly coupling through reductive amination or via a linker, such as adipic acid or squarate. As an alternative, the adjuvants may be non-covalently associated with additional agents, such as by way of hydrophobic or ionic interactions.

The contents of all of the above cited patents, patent applications and journal articles are incorporated by reference as if set forth fully herein.

Another embodiment provides a composition comprising: the compound synthesized according to the methods described herein and another agent. In some embodiments, the other agent is an immunogenic composition. In further embodiments, the agent is an antigen. In still further embodiments, the agent is a vaccine and the compound is a vaccine adjuvant. In another embodiment, the composition further comprises poly(lactide-co-glycolide) (PLG). In another embodiment, the composition further comprises MF59 or another adjuvant.

In another embodiment or method, the compound synthesized according to the methods described herein is administered topically to a subject.

Another embodiment provides a pharmaceutical composition, comprising: the compound synthesized according to the methods described herein and a pharmaceutically acceptable excipient.

In another embodiment, the compound synthesized according to the methods described herein is administered topically. More particularly the compound is administered topically to a lesion caused by a viral infection. More particularly the viral infection is Herpes simplex virus (HSV), more particular still, Type II Herpes simplex virus. In another embodiment the virus is human Papilloma virus (HPV). Alternatively, the compound synthesized according to the methods described herein is administered topically to a lesion caused by actinic keratosis.

Another embodiment of the present invention provides a method of stimulating TLR-7 production comprising administering a compound synthesized according to the methods described herein. Another embodiment provides a method of stimulating TLR-8 production comprising administering a compound synthesized according to the methods described herein. Another embodiment provides a method of stimulating TLR-7 and TLR-8 production comprising administering a compound synthesized according to the methods described herein.

Compounds of the present invention cause immune potentiation and stimulate production of TLR-7 and TLR-8. Such compounds can be used as polyclonal activators for the production of antigens. More particularly the invention relates to a method of preparing monoclonal antibodies with a desired antigen specificity comprising contacting the compounds of the present invention (such as those of formula I) with immortalized memory B cells.

The monoclonal antibodies produced therefrom, or fragments thereof may be used for the treatment of disease, for the prevention of disease or for the diagnosis of disease. Methods of diagnosis may include contacting an antibody or an antibody fragment with a sample. The methods of diagnosis may also include the detection of an antigen/antibody complex.

The memory B cells to be transformed can come from various sources (e.g. from whole blood, from peripheral blood mononuclear cells (PBMCs), from blood culture, from bone marrow, from organs, etc.), and suitable methods for obtaining human B cells are well known in the art. Samples may include cells that are not memory B cells or other blood cells. A specific human memory B lymphocyte subpopulation exhibiting a desired antigen specificity may be selected before the transformation step by using methods known in the art. In one embodiment, the human memory B lymphocyte subpopulation has specificity for a virus e.g. the B cells are taken from a patient who is suffering or has recovered from the virus. In another embodiment, B cells are taken from subjects with Alzheimer's disease and include B cells with specificity for B-amyloid (e.g. Mattson & Chan (2003) Science 301:1 847-9; etc.).

Another embodiment provides a method for producing immortalized B memory lymphocytes, comprising the step of transforming B memory lymphocytes using the Epstein Barr virus in the presence of a compound of the present invention, such as a compound synthesized according to the methods described herein. See WO 04/76677.

The invention also provides pharmaceutical compositions that include any of the aforementioned compounds or embodiments of formula I. Such compositions may include other pharmaceutically acceptable ingredients such as one or more of excipients, carriers, and the like well-known to those skilled in the art.

The imidazoquinoline compounds can be used with or without an antigen in therapeutic applications, for example to treat cancer or infectious diseases. The imidazoquinoline compounds may also be used in combination with other therapeutic agents, such as anti-viral agents and monoclonal antibodies in different therapeutic applications.

One embodiment of the method of inducing an immunostimulatory effect in a patient is directed to administering an immunogenic composition comprising a vaccine in an amount effective to stimulate an immune response such as a cell-mediated immune response and, as a vaccine adjuvant, an imidazoquinoline compound, in an amount effective to potentiate the immune response such as the cell-mediated immune response to the vaccine.

### DEFINITIONS

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Acyl includes the “acetyl” group CH₃C(O)—.

“Acylamino” refers to the groups —NRC(O)alkyl, —NRC(O)substituted alkyl, —NRC(O)cycloalkyl, —NRC(O)substituted cycloalkyl, —NRC(O)cycloalkenyl, —NRC(O)substituted cycloalkenyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R′ and R″ are optionally joined, together with the nitrogen bound thereto to form a heterocyclic or substituted heterocyclic group, provided that R′ and R″ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonylamino” refers to the group —NRC(O)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonylamino” refers to the group —NRC(S)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonylamino” refers to the group —NR—SO₂NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkyenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkyenyl, aryl, substituted aryl, heteroaryl, substituted heteroaryl, heterocyclic and substituted heterocyclic are as defined herein.

“Amidino” refers to the group —C(═NR¹²)R¹⁰R¹¹ where R¹⁰, R¹¹, and R¹² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Alkenyl” refers to alkenyl groups having from 2 to 6 carbon atoms and preferably 2 to 4 carbon atoms and having at least 1 and preferably from 1 to 2 sites of alkenyl unsaturation. Such groups are exemplified, for example, by vinyl, allyl, and but-3-en-1-yl.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to a vinyl (unsaturated) carbon atom.

“Alkynyl” refers to alkynyl groups having from 2 to 6 carbon atoms and preferably 2 to 3 carbon atoms and having at least 1 and preferably from 1 to 2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to an acetylenic carbon atom.

“Carbonyl” refers to the divalent group —C(O)— which is equivalent to —C(═O)—.

“Carboxyl” or “carboxy” refers to —COOH or salts thereof

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, substituted —NR—C(O)O-alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, substituted —O—C(O)O-alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, substituted alkyl, aryl, substituted aryl, heteroaryl, substituted heteroaryl, heterocyclic, and substituted heterocyclic and two R¹³ groups attached to a common guanidino nitrogen atom are optionally joined together with the nitrogen bound thereto to form a heterocyclic or substituted heterocyclic group, provided that at least one R¹³ is not hydrogen, and wherein said substituents are as defined herein.

“Halo” or “halogen” refers to fluoro, chloro, bromo and iodo.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or unsaturated group having a single ring or multiple condensed rings, including fused bridged and spiro ring systems, from 1 to 10 carbon atoms and from 1 to 4 hetero atoms selected from the group consisting of nitrogen, sulfur or oxygen within the ring wherein, in fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spirocycloalkyl” refers to divalent cyclic groups from 3 to 10 carbon atoms having a cycloalkyl ring with a spiro union (the union formed by a single atom which is the only common member of the rings) as exemplified by the following structure:

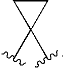

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N-moiety such as pyrazoles, imidazoles, benzimidazoles, triazoles, and tetrazoles.

“Reacting” refers to modifying conditions such that an unreactive molecule becomes reactive. This may involve addition of solvent(s), a catalyst, reagents, coupling agents, and/or heat, among others.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

The term “protected” or a “protecting group” with respect to hydroxyl groups, amine groups, and sulfhydryl groups refers to forms of these functionalities which are protected from undesirable reaction with a protecting group known to those skilled in the art such as those set forth in Protective Groups in Organic Synthesis, Greene, T. W., John Wiley & Sons, New York, N.Y., (1st Edition, 1981) which can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, benzyl or dibenzyl, amides such as, formamide, acetamide, trifluoroacetamide, and benzamide; imides, such as phthalimide, and dithiosuccinimide; and others. In some embodiments, a protecting group for amines is a benzyl group. Examples of protected sulfhydryl groups include, but are not limited to, thioethers such as S-benzyl thioether, and S-4-picolyl thioether; substituted S-methyl derivatives such as hemithio, dithio and aminothio acetals; and others.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

The foregoing may be better understood by reference to the following Examples that are presented for illustration and not to limit the scope of the inventive concepts. The Example compounds and their analogs are easily synthesized by one skilled in the art from procedures described herein, as well as in patents or patent applications listed herein which are all hereby incorporated by reference in their entireties and for all purposes as if fully set forth herein.

### EXAMPLES

Referring to the examples that follow, compounds of the preferred embodiments were synthesized using the methods described herein, or other methods, which are known in the art.

The compounds and/or intermediates were characterized by high performance liquid chromatography (HPLC) using a Waters Millenium chromatography system with a 2695 Separation Module (Milford, Mass.). The analytical columns were reversed phase Phenomenex Luna C18-5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on one of two LCMS instruments: a Waters System (Alliance HT HPLC and a Micromass ZQ mass spectrometer; Column: Eclipse XDB-C18, 2.1×50 mm; gradient: 5-95% (or 35-95%, or 65-95% or 95-95%) acetonitrile in water with 0.05% TFA over a 4 min period; flow rate 0.8 mL/min; molecular weight range 200-1500; cone Voltage 20 V; column temperature 40° C.) or a Hewlett Packard System (Series 1100 HPLC; Column: Eclipse XDB-C18, 2.1×50 mm; gradient: 5-95% acetonitrile in water with 0.05% TFA over a 4 min period; flow rate 0.8 mL/min; molecular weight range 150-850; cone Voltage 50 V; column temperature 30° C.). All masses were reported as those of the protonated parent ions.

GCMS analysis is performed on a Hewlett Packard instrument (HP6890 Series gas chromatograph with a Mass Selective Detector 5973; injector volume: 1 μL; initial column temperature: 50° C.; final column temperature: 250° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis was performed on some of the compounds with a Varian 300 MHz NMR (Palo Alto, Calif.). The spectral reference was either TMS or the known chemical shift of the solvent. Some compound samples were run at elevated temperatures (e.g., 75° C.) to promote increased sample solubility.

The purity of some of the compounds is assessed by elemental analysis (Desert Analytics, Tucson, Ariz.).

Melting points are determined on a Laboratory Devices MeI-Temp apparatus (Holliston, Mass.).

Preparative separations are carried out using a Flash 40 chromatography system and KP-Sil, 60A (Biotage, Charlottesville, Va.), or by flash column chromatography using silica gel (230-400 mesh) packing material, or by HPLC using a Waters 2767 Sample Manager, C-18 reversed phase column, 30×50 mm, flow 75 mL/min. Typical solvents employed for the Flash 40 Biotage system and flash column chromatography are dichloromethane, methanol, ethyl acetate, hexane, acetone, aqueous ammonia (or ammonium hydroxide), and triethyl amine. Typical solvents employed for the reverse phase HPLC are varying concentrations of acetonitrile and water with 0.1% trifluoroacetic acid.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

The examples below as well as throughout the application, the following abbreviations have the following meanings. If not defined, the terms have their generally accepted meanings.

### Abbreviations

ACN Acetonitrile
BINAP 2,2′-bis(diphenylphosphino)-1,1′-binapthyl
DCM Dichloromethane
DIEA diisopropylethylamine
DIPEA N,N-diisopropylethylamine
DME 1,2-dimethoxyethane
DMF N,N-dimethylformamide
DMSO dimethyl sulfoxide
DPPF 1,1′-bis(diphenylphosphino)ferrocene
EtOAc ethyl acetate
EtOH ethanol
HATU 2-(7-Aza-1H-benzotriazole-1-yl)-1,1,3,3-tetramethyluronium hexafluorophosphate
HPLC high performance liquid chromatography
MCPBA meta-chloroperoxybenzoic acid
MeOH methanol
NBS N-bromosuccinimide
NMP N-methyl-2-pyrrolidone
RT room temperature
THF tetrahydrofuran

### Example 1

### General Routes for Preparation of Compounds According to the Methods of the Invention

Schemes I, II and III, below, describe the preparation of some preferred compounds according to the methods of the invention.

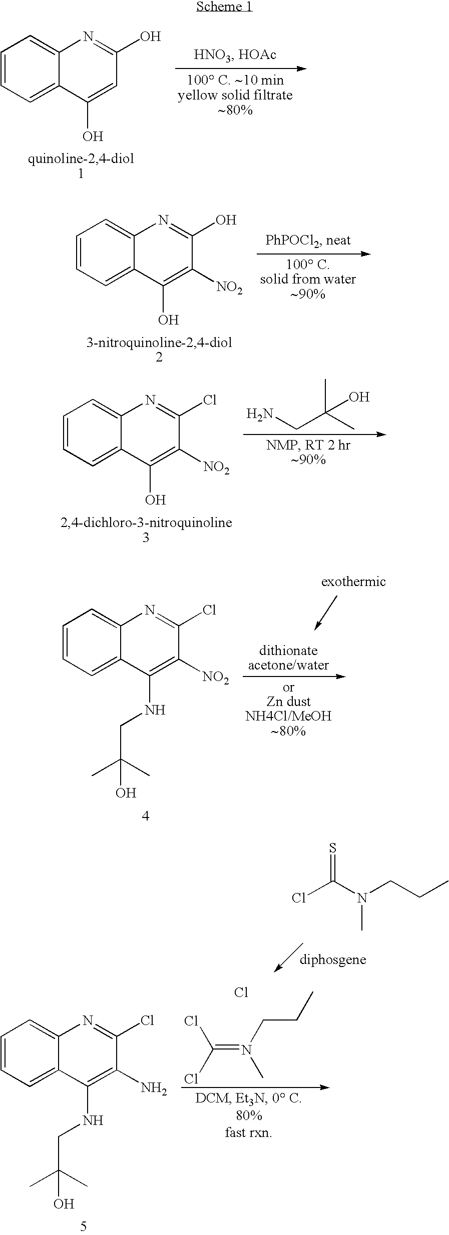

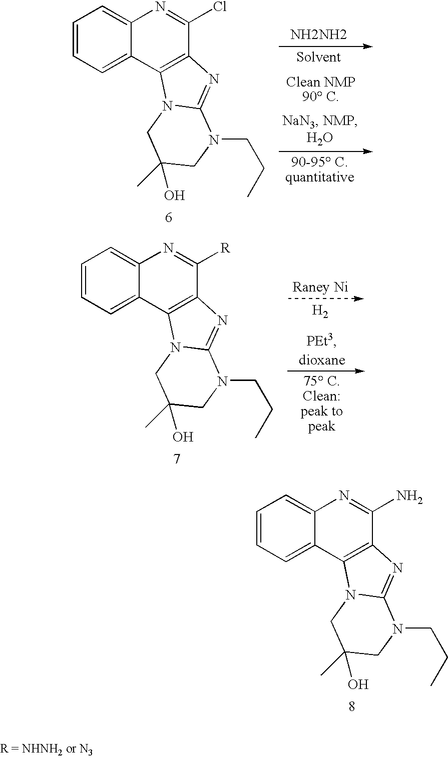

In accordance with Scheme 1, quinoline-2,4-diol 1 is nitrated with nitric acid in acetic acid to yield 3-nitroquinoline-2,4-diol 2. Chlorination with phenylphosphonic dichloride yields 2,4-dichloro-3-nitroquinoline 3. Reaction with 2-methylaminoisopropylalcohol yields 2-chloro-3-nitro-4-(2-hydroxy-2-methyl-propylamino) quinoline 4. Subsequent reduction of the nitro group provides the corresponding 3,4-diamino compound 5. Reaction of 5 with a dichloro immonium compound of general formula Cl₂C═N(R′)(R″), prepared from the reaction of ClC(═S)N(R′)(R″) with diphosgene, yields the substituted 4-chloroimidazoquinoline 6. Displacement of halogen with hydrazine or azide yields the corresponding hydrazide or azide 7, and subsequent reduction provides the final amino compound 8.

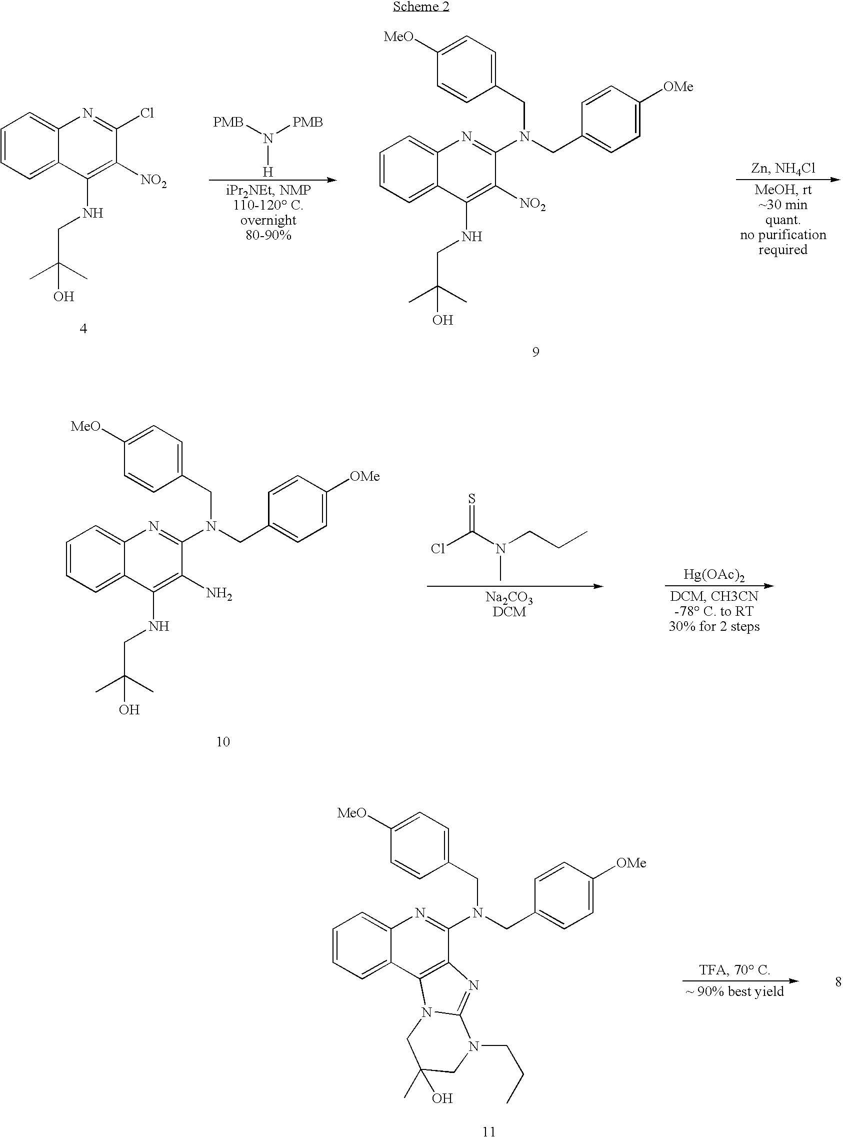

In accordance with Scheme 2, displacement of halogen from 2-chloro-3-nitro-4-(2-hydroxy-2-methyl-propylamino) quinoline 4 with NH(PMB)₂ yields the protected amino compound 9. Reduction of the nitro group provides the corresponding amino compound 10, which is then reacted with ClC(═S)N(R′)(R″) and Hg(OAc)₂ to provide the protected imidazoquinoline compound II. Subsequent removal of the p-methoxybenzyl protecting groups provides the final amino compound 8.

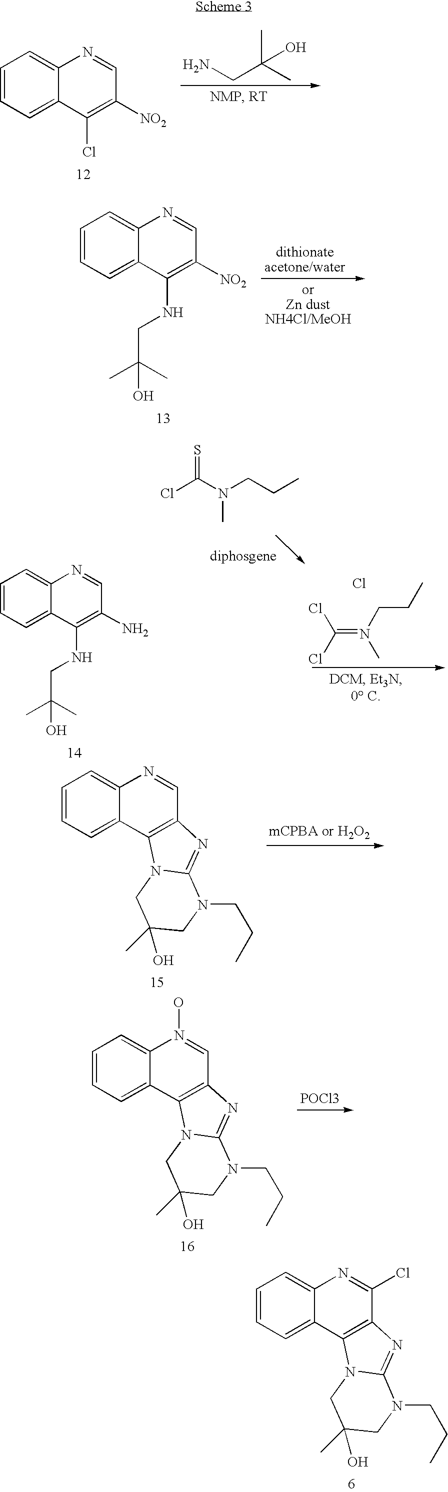

In accordance with Scheme 3,3-nitro-4-chloroquinoline 12 is reacted with 2-methylaminoisopropylalcohol yields 3-nitro-4-(2-hydroxy-2-methyl-propylamino) quinoline 13. Subsequent reduction of the nitro group yields the corresponding amino compound 14. Reaction of 14 with a dichloro immonium compound of general formula Cl₂C═N(R′)(R″), prepared from the reaction of ClC(═S)N(R′)(R″) with diphosgene, yields the substituted imidazoquinoline 15. Oxidation of the quinoline nitrogen to the N-oxide, followed by halogenation with POCl₃, yields the corresponding 4-chloro compound 6, which can be treated as described above in Scheme I.

Scheme 4, below, summarizes some routes of the methods of the invention to the preparation of imidazole quinolines.

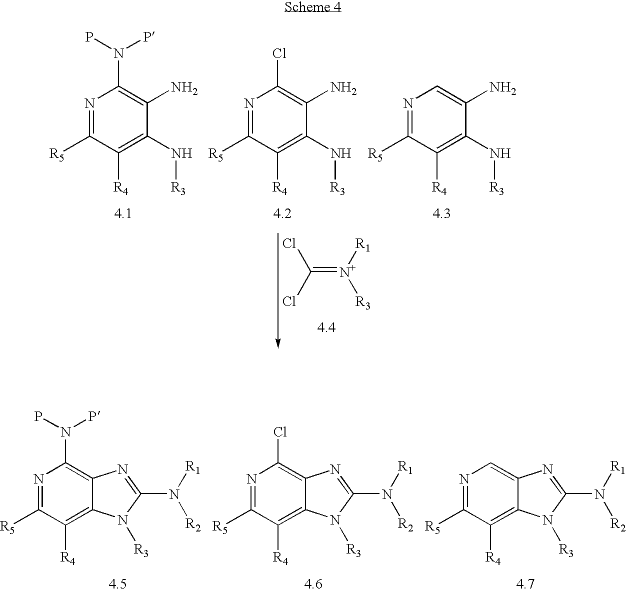

Scheme 4 describes how intermediates of formulas 4.1-4.3, which are precedented in the literature or can be prepared following procedures described herein, can be transformed to intermediates 4.5-4.7, respectively, by treating the diamino intermediates 4.1-4.3 with an iminium reagent such as, for example, the intermediate of formula 4.4, which are precedented in the literature or can be prepared following procedures described herein. Intermediates of formulas 4.5 and 4.7 can be transformed to compounds of the embodiment through methods described previously. Intermediates of formula 4.6 can be taken on to compounds of the embodiment by displacement of the chloride with a suitably substituted amine to obtain intermediates of formula 4.5. Additionally, intermediates of formula 4.6 can be taken to compound of the embodiment by displacement with, for example, an azide, hydrazide or hydroxylamine followed by reduction by methods, which can be readily found by one trained in the art.

### Preparation of Compounds According to Methods of the Invention

### Example 2

### 3-nitroquinoline-2,4-diol

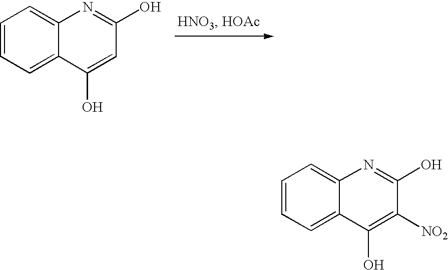

The title compound was prepared following methods described by Buckle, Derek R.; Cantello, Barrie C. C.; Smith, Harry; Spicer, Barbara A. 4-Hydroxy-3-nitro-2-quinolones and related compounds as inhibitors of allergic reactions. Journal of Medicinal Chemistry 1975, 18(7), 726-32, incorporated herein by reference in its entirety.

In a 500 mL round bottom flask was added quinoline-2,4-diol (16.2 g, 0.1 mol) followed by glacial HOAc (100 mL, 1.74 mol) and HNO₃ (70%, 26 mL, 0.4 mol). The reaction remained a suspension and thickened to the point where stirring was not possible. The liquid portion was dark brown and the solid appeared off-white at this time. The reaction vessel was fitted with a reflux condenser (securely clipped), placed in an oil bath (105° C.) and rotated slowly by hand for ˜5-8 minutes at which point the off-white solid completely dissolved (dark brown liquid). Heating/rotation was continued and a yellow solid began to form (˜30 sec.-1 min. after dissolution). This solid continued to form until the reaction mixture could no longer be stirred. Heating was continued for ˜2 min. The reaction was then cooled to room temperature and water (˜100 mL) was added. The solid was broken up manually and collected by filtration. The solid was washed liberally with water and then diethyl ether, and then dried under vacuum. The above reaction was repeated three times on a total of 48.6 g (0.3 mol) to provide a combined yield of 49 g (79%) of the title compound. HPLC t_{R}=1.73 min; LCMS m/z=207.0, t_{R}=1.67 min (MH⁺); ¹H NMR (300 MHz, DMSO): δ 11.95 (s, 1H), 8.01 (dd, 1H), 7.63 (m, 1H), 7.31 (d, 1H), 7.25 (m, 1H); ¹³C NMR (75 MHz, DMSO): δ 157.0, 156.5, 138.8, 133.8, 127.9, 125.2, 123.0, 116.5, 114.8.

### Example 3

### 2,4-dichloro-3-nitroquinoline

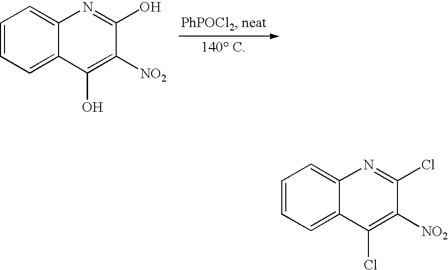

The title compound was prepared following procedure outlined by Izumi, Tomoyuki; Sakaguchi, Jun; Takeshita, Makoto; Tawara, Harumi; Kato, Ken-Ichi; Dose, Hitomi; Tsujino, Tomomi; Watanabe, Yoshinari; Kato, Hideo. 1H-Imidazo[4,5-c]quinoline derivatives as novel potent TNF-α suppressors: synthesis and structure-activity relationship of 1-, 2- and 4-substituted 1H-imidazo[4,5-c]quinolines or 1H-imidazo[4,5-c]pyridines. Bioorganic & Medicinal Chemistry 2003, 11(12), 2541-2550, incorporated herein by reference in its entirety.

3-Nitroquinoline-2,4-diol (13.4 g, 65 mmol) and phenylphosphonic dichloride (41 mL, 260 mmol) were combined at room temperature under nitrogen and then heated to 140° C. for 3 hours. The mixture was poured into ice water and stirred vigorously for 30 minutes, and filtered to capture the solid formed. The solid was rinsed twice with water and then dried overnight under vacuum to provide 2,4-dichloronitroquinoline (13.2 g). HPLC t_{R}=4.69 min; LCMS m/z=243:245:247=9:6:1, t_{R}=333 min (MH⁺); ¹H NMR (300 MHz, CDCl₃): δ 8.27 (m, 1H), 8.11 (m, 1H), 7.95 (m, 1H), 7.81 (m, 1H); ¹³C NMR (75 MHz, CDCl₃): δ 146.8 (2C), 139.9, 135.8, 133.7, 130.0, 129.6, 125.4, 126.7; ¹H NMR (300 MHz, DMSO): δ 8.32 (m, 1H), 8.08-8.17 (m, 2H), 7.97 (m, 1H); ¹³C NMR (75 MHz, DMSO): δ 146.1 (2C), 138.2, 135.5, 134.3, 130.6, 128.9, 125.3, 123.9.

### Example 4

### 1-(2-chloro-3-nitroquinolin-4-ylamino)-2-methylpropan-2-ol

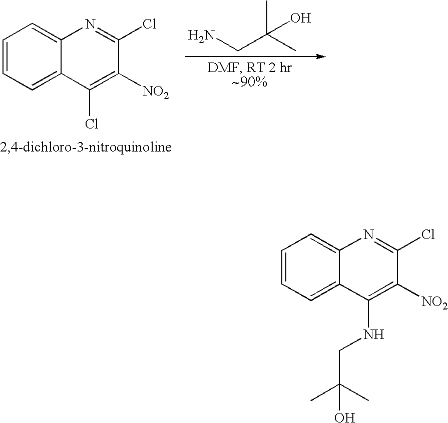

To a room temperature solution of 2,4-dichloro-3-nitro quinoline (˜94% pure, 17.9 g, 73.6 mmol) in DMF (100 mL) was added triethylamine (20.4 mL, 146.8 mmol), 4 Å mol. sieves (10 g) and lastly 1-amino-2-methylpropan-2-ol (6.86 g in 10 mL DMF, 77.0 mmol). The reaction mixture was stirred at room temperature for ˜3 hours. HPLC indicated SM consumed and product formed cleanly. Reaction mixture was transferred to a separatory funnel, diluted with ethyl acetate (500 mL) and washed twice with water:brine (3:1, 400 mL). Aqueous layers were back extracted once with ethyl acetate. Combined organics were dried over MgSO₄, filtered and concentrated. Solid was triturated with diethyl ether (˜200 mL) and sonicate. The solid was collected by filtration, rinsed with minimum of ether and dried under vacuum to provide the desired product (16.8 g). HPLC t_{R}=3.75 min; LCMS m/z=296:298=3:1, t_{R}=2.75 min (MH⁺); ¹H NMR (300 MHz, CDCl₃): δ 7.92 (m, 2H), 7.74 (m, 1H), 7.54 (m, 1H), 6.51 (brs., 1H), 3.28 (d, 2H), 1.74 (brs., 1H), 1.34 (s, 6H); ¹H NMR (300 MHz, DMSO): δ 8.33 (d, 1H), 7.81-7.85 (m, 2H), 7.65 (m, 1H), 7.25 (t., 1H), 5.00 (s, 1H), 3.09 (d, 2H), 1.13 (s, 6H); ¹³C NMR (75 MHz, DMSO): δ 145.6, 145.4, 141.0, 132.4, 128.6, 126.8, 126.6, 123.2, 119.4, 69.0, 54.2, 27.1.

### Example 5

### Bis(4-methoxybenzyl)amine

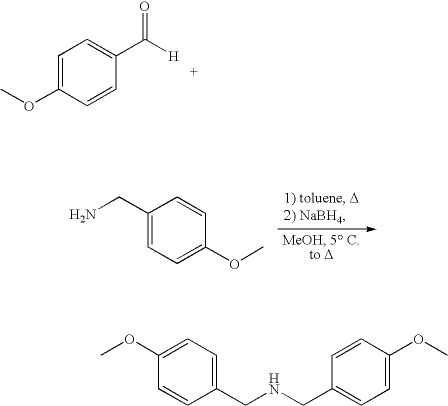

p-Anisaldehyde (25.0 g, 0.1836 mol), 4-methoxybenzylamine (25.3 g, 0.1836 mol) and toluene (150 mL) were combined in a 500 mL round bottom flask which was fitted with a condenser and Dean-Stark trap under a N₂ atmosphere. The solution was refluxed for 3 hours during which time 3 mL of H₂O was azeotroped away from the reaction mixture. The reaction was cooled and concentrated on a rotovap at 40° C. for 2 hours. The clear, yellow oil was taken up in MeOH (150 mL) in a 500 mL round bottom flask fitted with a condenser under a N₂ atmosphere. The reaction was cooled to 5° C., and NaBH₄ was added in small portions over 45 min (off-gassing occurred). The reaction was slowly heated to reflux with vigorous off-gassing. After 2 hours at reflux, the reaction was cooled to room temperature and concentrated on the rotorvap at 30° C. for 2 hours, and then placed under high vacuum at 30° C. for 1 hour to give the title compound as a white crystalline solid (47.13 g, quantitative yield; 98.6% purity by HPLC). MH⁺=258.1

### Example 6

### 1-(2-(Bis(4-methoxybenzyl)amino)-3-nitroquinolin-4-ylamino)-2-methylpropan-2-ol

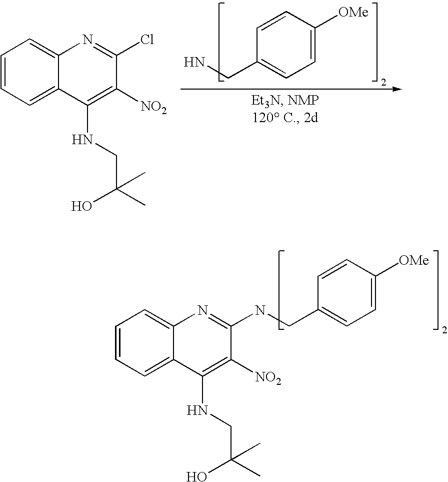

1-(2-chloro-3-nitroquinolin-4-ylamino)-2-methylpropan-2-ol (5.01 g, 17.0 mmol), bis(4-methoxybenzyl)amine (MM-17594-128-1, 6.02 g, 23.4 mmol), triethylamine (7.1 mL, 50.1 mmol) and NMP (7.5 mL) were combined in glass bomb. The reaction was heated at 120° C. for 2 days. HPLC indicated the reaction went to 95% completion. The reaction mixture was combined with three reactions mixtures run previously, and this combined material was taken up in CH₂Cl₂. The organic layer was washed with H₂O (2×), 0.5M citrate (2×), H₂O and brine and then dried over Na₂SO₄, filtered and concentrated to a red gum (18.30 g). The crude material was purified by column chromatography (0-50% EtOAc/Hexanes) to give the title compound as a red syrup (10.1 g, 82% yield). MH⁺ 258.1

### Example 7

### 1-(3-amino-2-(bis(4-methoxybenzyl)amino)quinolin-4-ylamino)-2-methylpropan-2-ol

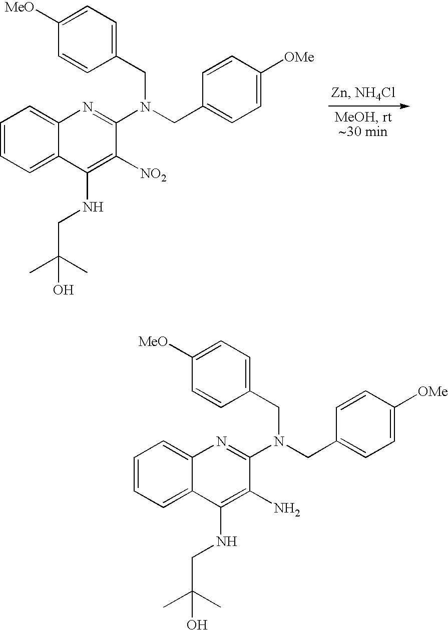

To a solution of nitro compound 1-(2-(Bis(4-methoxybenzyl)amino)-3-nitroquinolin-4-ylamino)-2-methylpropan-2-ol (˜8 g) in methanol (75 mL) was added Zn dust (5.16 g, 79.5 mmol) followed by ammonium chloride (5.16 g, 97.3 mmol). The reaction was sonicated while swirling by hand for ˜2 minutes and then stirred at room temperature for ˜20 minutes. An additional portion of Zn (1.16 g, 17.8 mmol) and ammonium chloride (1.16 g, 21.9 mmol) was added and stirring continued for 20 minutes. A predominance of yellow color/brown color disappeared after the second addition of reagents. The reaction was filtered through celite and the celite was washed liberally with methanol until the eluent showed no UV activity detected on TLC. Solvent was removed under vacuum and the residue was taken up in 30% methanol in dichloromethane. Solids were removed by filtration and then solvent was removed under vacuum. Purification by flash chromatography (120 g ISCO silica cartridge, 0-30% methanol in dichloromethane, 20 min. grad, 85 mL/min) provided the title compound (7.8 g, 16.5 mmol). MH⁺=487.2

### Example 8

### Methyl(propyl)carbamothioic chloride

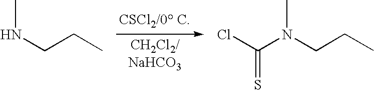

To a round bottom flask fitted with an addition funnel was added N-methylpropan-1-amine (10.2 g, 0.139 mole) and sodium bicarbonate (35.12 g, 0.417 mole) followed by methylene chloride (400 ml). The flask was cooled to 0° C. with ice. Thiophosgene (13.86 ml, 0.180 mole) was added drop-wise to the round bottom flask. The reaction mixture was then stirred for 0.5 hour at 0° C. and then brought to ambient temperature and stirred for another 0.5 hour. The reaction mixture was monitored by TLC (30% ethyl acetate/hexane, and developed with iodine) and starting material was consumed to give methyl(propyl)carbamothioic chloride. The reaction mixture was washed with water followed by saturated sodium chloride solution (3 times) and the organic layer was dried with sodium bicarbonate and concentrated to a pale yellow oil and dried under high vacuum. 18.6 g (92% recovery) of methyl(propyl)carbamothioic chloride were obtained.

### Example 9

### 1-(4-(bis(4-methoxybenzyl)amino)-2-(methyl(propyl)amino)-1H-imidazo[4,5-c]quinolin-1-yl)-2-methylpropan-2-ol

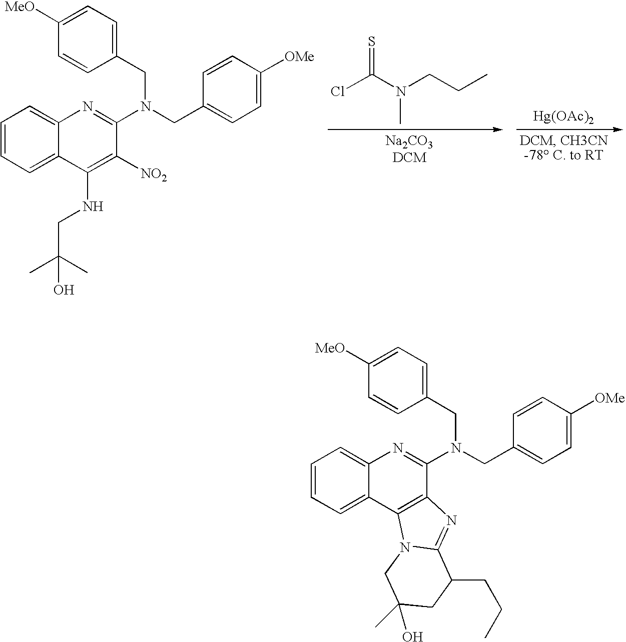

To a solution of crude 1-(3-amino-2-(bis(4-methoxybenzyl)amino)quinolin-4-ylamino)-2-methylpropan-2-ol (20 mmol) in dichloromethane (350 mL) at room temperature was solid sodium carbonate (8.5 g, 80 mmol) followed by methyl(propyl)carbamothioic chloride (4.5 g, 30 mmol). The reaction was stirred overnight at room temperature. LCMS indicated starting material, mono-addition product and bis-addition product were present. An additional portion of methyl(propyl)carbamothioic chloride (1.5 g, 9.9 mmol) was added and the reaction was stirred for an additional 3 hours at which time the LCMS indicated starting material had been consumed and the primary product was the bis-methyl(propyl)carbamothioic chloride addition product. Acetonitrile (100 mL) was added and the reaction mixture was cooled to −78° C. and Hg(OAc)₂ (16 g, 50 mmol) was added as a solid. The reaction mixture was stirred at −78° C. for 20 min., the cooling bath was removed and the reaction was allowed to warm to room temperature wile stirring. Reaction mixture was stirred at room temperature for ˜30 minutes. Solvent was removed under vacuum and the residue was taken up in acetonitrile (150 mL) and filtered to remove solids. Solvent was removed under vacuum to dryness. Purification by flash chromatography (silica gel, 0-40% ethyl acetate in hexanes, step gradient 0-10-20-30-40% by hand, identify product by TLC 40% ethyl acetate in hexanes, Rf=0.7, fluorescent on TLC under UV) provided the title compound (3.2 g, 5.6 mmol). MH⁺=568.2

### Example 10

### 1-(4-amino-2-(methyl(propyl)amino)-1H-imidazo[4,5-c]quinolin-1-yl)-2-methylpropan-2-ol

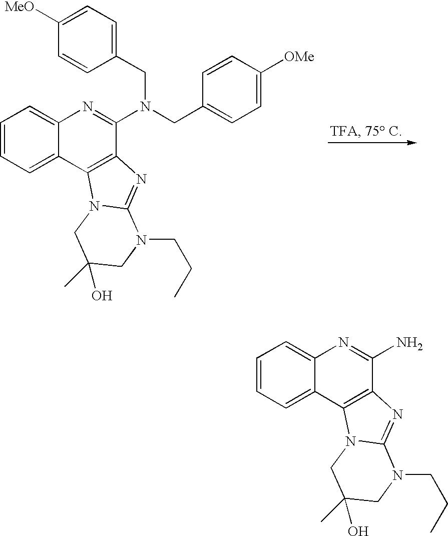

1-(4-(bis(4-methoxybenzyl)amino)-2-(methyl(propyl)amino)-1H-imidazo[4,5-c]quinolin-1-yl)-2-methylpropan-2-ol (2.0 g, 3.53) was taken up in TFA (35 mL). The reaction mixture was heated to 75° C. for ˜6 hours. The light brown reaction mixture was cooled to room temperature and diethyl ether (150 mL) was added to provide a tan precipitate. The solid was collected by filtration and washed with a minimum of diethyl ether. The solid was partitioned in an Erlenmyer flask between water (50 mL) and ethyl acetate (100 mL). Saturate aqueous sodium bicarbonate was added carefully (50 mL) and the mixture was stirred at room temperature for 20 minutes. The mixture was transferred to a separatory funnel and the organic phase was isolated. The aqueous layer was extracted twice more with ethyl acetate. The combined organics were dried over MgSO₄, filtered and concentrated. The residue was taken up in methanol:ethylacetate (1:1) and silica gel (˜15 g) was added. Solvents were removed under vacuum and the solid dried under vacuum overnight. The product loaded silica gel was carefully added to the top of a silica gel column (10 cm dia. by 50 cm, wet load to column with hexane). The product loaded silica gel was carefully wetted with hexane, minimizing agitation, and then sand was loaded to the top of the column. Elution was begun e with 1:5:14 methanol:ethylacetate:hexane until product began to elute (TLC) and then continued with 1:3:6 methanol:ethylacetate:hexane until product completely eluted. The desired fractions were combined solvent removed until ˜15 mL volume remained. Trituration with diethyl ether (75 ml) and then hexane (25 mL), followed by collection of solid by filtration and drying under vacuum overnight provided the title compound (1.16 g, 3.53 mmol). MH⁺=328

### Example 11

### 1-(3-amino-2-chloroquinolin-4-ylamino)-2-methylpropan-2-ol

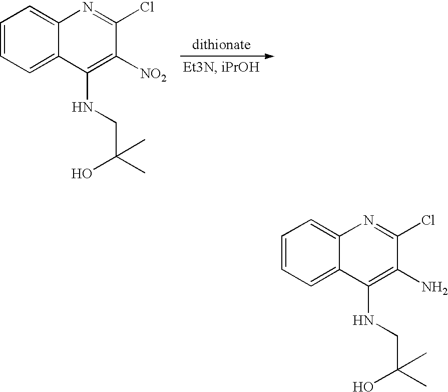

To a solution of 1-(2-chloro-3-nitroquinolin-4-ylamino)-2-methylpropan-2-ol (5.0 g, 16.9 mmol) in iPrOH (30 mL) was added triethylamine (17 mL, 12.3 g, 122 mmol) followed by water (40 mL). The reaction mixture was cooled to 0° C. and then a solution of Na₂S₂O₄ (19.5 g, 111.9 mmol) in water (80 mL) was added dropwise via dropping funnel over 40 minutes while retaining cooling at 0° C. Reaction mixture was then stirred at 0° C. for 30 minutes. Conc. HCl (20 mL) was then added and the resulting mixture transferred to a separatory funnel and washed with ethyl acetate (200 mL). The ethyl acetate layer was then extracted with 3M HCl (50 mL). The combined aqueous extracts were then taken to pH ˜7 with addition of K₃PO₄ (˜41 g). The resulting mixture was then extracted with diethyl ether (2×300 mL). The combined ether extracts were washed once with brine, dried over MgSO₄, filtered and concentrated. Purification by flash chromatography (silica gel, ethyl acetate/hexane) provided the title compound (3.2 g, 71.5%). HPLC t_{R}=1.76 min; LCMS m/z=266:268=3:1, t_{R}=1.77 min (MH⁺); ¹H NMR (300 MHz, CDCl₃): δ 7.87-7.91 (m, 1H), 7.78-7.81 (m, 1H), 7.40-7.50 (m, 2H), 4.20-4.40 (m, 3H), 3.20 (d, 2H), 2.13 (brs., 1H), 1.39 (s, 6H); ¹³C NMR (75 MHz, CDCl₃): δ 142.4(2C), 137.2, 129.3, 128.7, 126.8, 126.4, 123.6, 120.2, 71.5, 56.8, 27.8;

¹H NMR (300 MHz, DMSO): δ 8.04-8.04 (m, 1H), 7.66-7.70 (m, 1H), 7.39-7.45 (m, 2H), 5.13 (brs., 2H), 5.08 (t, 1H), 4.82 (s, 1H), 3.18 (d, 2H), 1.15 (s, 6H); ¹³C NMR (75 MHz, DMSO): δ 141.13, 141.07, 137.7, 128.0, 127.8, 125.8, 125.0, 122.5, 122.0, 69.9, 57.3, 27.3.

### Example 12

### N-(dichloromethylene)-N-methylpropan-1-aminium chloride

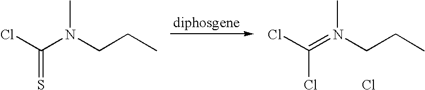

The title compound was prepared by adding over 50 minutes a solution of diphosgene (1.47 g, 7.5 mmol) in dichloromethane (6 mL) to a solution of methyl(propyl)carbamothioic chloride (1.51 g, 10 mmol) in dichloromethane (6 mL). The resulting mixture was then refluxed for 3 hours. Hexane (15 mL) was added and the reaction mixture was cooled to 0° C. The resulting solid was collected by filtration under an inert atmosphere (nitrogen flow) to provide the title compound (835 mg, 44%), which was immediately taken up in dichloromethane for the subsequent reaction.

### Example 13

### 1-(4-chloro-2-(methyl(propyl)amino)-1H-imidazo[4,5-c]quinolin-1-yl)-2-methylpropan-2-ol

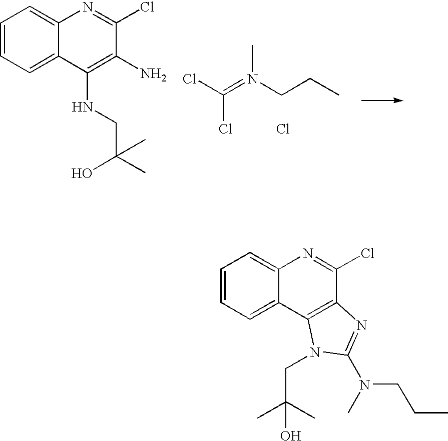

To a solution of 1-(3-amino-2-chloroquinolin-4-ylamino)-2-methylpropan-2-ol (580 mg, 2.19 mmol) in dichloromethane (2 mL) was added triethylamine (774 mg, 7.67 mmol). The solution was cooled to 0° C. and then a solution of N-(dichloromethylene)-N-methylpropan-1-aminium chloride (562 mg, 2.95 mmol) in dichloromethane (18 mL) was added dropwise over 10-15 minutes while retaining the temperature at 0° C. and was then stirred at 0° C. for 30 minutes. The reaction mixture was diluted with ethyl acetate (150 mL), transferred to a separatory funnel and washed with brine (1×). The organics were then dried over MgSO₄, filtered and concentrated. Purification by flash chromatography (silica gel, ethyl acetate/hexane (2:3)) provided the title compound (610 mg, 80.5%). HPLC t_{R}=3.04 min; LCMS m/z=347:349=3:1, t_{R}=2.50 min (MHe); ¹H NMR (300 MHz, CDCl₃): δ 8.26 (m, 1H), 8.11 (m, 1H), 7.58 (m, 2H), 4.66 (s, 2H), 3.16-3.21 (m, 3H), 2.98 (s, 3H), 1.68-1.76 (m, 2H), 1.19 (s, 6H), 1.00 (t, 3H); ¹³C NMR (75 MHz, CDCl₃): δ 159.7, 144.0, 143.0, 135.0, 132.2, 130.2, 127.3, 126.0, 120.3, 117.9, 57.9, 55.9, 40.1, 27.7, 20.6, 11.5; ¹H NMR (300 MHz, DMSO): δ 8.56-8.60 (m, 1H), 7.92-7.96 (m, 1H), 7.56-7.62 (m, 2H), 4.51 (brs., 2H), 3.10 (t, 2H), 2.87 (s, 3H), 1.64 (m, 2H), 1.06 (brs., 6H), 0.92 (t, 3H). ¹³C NMR (75 MHz, DMSO): δ 160.3, 142.9, 141.2, 135.9, 131.4, 128.6, 126.8, 125.2, 123.1, 118.3, 71.0, 56.7, 55.2, 39.4, 27 (very broad), 19.9, 11.3.

### Example 14

### 1-(4-azido-2-(methyl(propyl)amino)-1H-imidazo[4,5-c]quinolin-1-yl)-2-methylpropan-2-ol

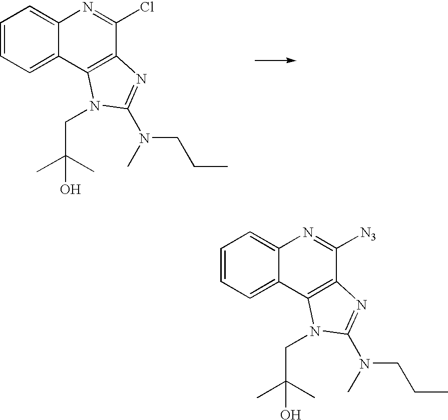

To a solution 1-(4-chloro-2-(methyl(propyl)amino)-1H-imidazo[4,5-c]quinolin-1-yl)-2-methylpropan-2-ol (0.8 g, 2.3 mmol) in NMP (12 mL) at room temperature was added sodium azide (1.5 g, 23 mmol). With stirring, water was added dropwise until mixture was lightly cloudy (˜5-7 mL). The reaction was then heated to 95° C. for 60 hours. The reaction was cooled to room temp. and water (50 mL) was added. The reaction was stirred for 2 hours at room temperature. The solid present was collected by filtration and washed with water (1×). The solid was dried under vacuum to provide the title compound (0.67 g).

HPLC t_{R}=3.23 min; LCMS m/z=354, t_{R}=2.57 min; ¹H NMR (300 MHz, CDCl₃): δ 8.74-8.78 (m, 1H), 8.38-8.42 (m, 1H), 7.66-7.73 (m, 2H), 4.65 (s, 2H), 3.17 (m, 2H), 2.97 (s, 3H), 2.81 (s, 1H), 1.67-1.75 (m, 2H), 1.23 (s, 6H), 0.97 (t, 3H); ¹³C NMR (75 MHz, CDCl₃): δ160.5, 143.4, 128.9, 128.3, 127.8, 127.1, 124.4, 122.3, 118.3, 116.2, 72.6, 58.1, 55.6, 40.3, 27.8, 20.5, 11.5.

### Example 15

### 1-(4-amino-2-(methyl(propyl)amino)-1H-imidazo[4,5-c]quinolin-1-yl)-2-methylpropan-2-ol

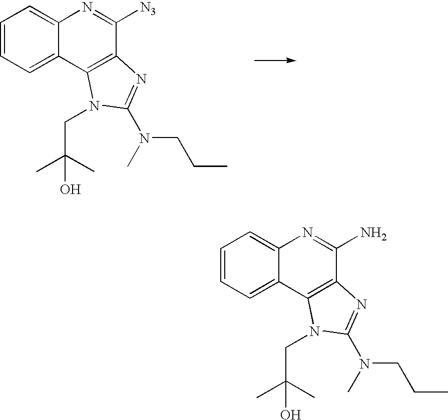

To a suspension of 1-(4-azido-2-(methyl(propyl)amino)-1H-imidazo[4,5-c]quinolin-1-yl)-2-methylpropan-2-ol (0.67 g, 1.90 mmol) in dioxane (12 mL) at room temperature was added PEt₃ (1.4 mL). The reaction was then heated to 70° C. overnight. HPLC indicated that the starting material had been consumed. Methanol (5 mL) and water (5 mL) were added to the reaction mixture and the reaction mixture was heated at 70° C. overnight. The reaction mixture was cooled to room temperature, diluted with ethyl acetate (45 mL) and washed with twice with saturated sodium bicarbonate. The aqueous washings were back extracted once with ethyl acetate. The combined organics were dried over sodium sulfate, filtered and concentrated to provide an off white solid. The solid was then triturated with ethyl acetate (once solid the product does not readily go into ethyl acetate) and the solid was collected by filtration and dried under vacuum to provide the title compound (0.53 g). The mother liquor was allowed to sit at room temperature and additional title compound crystallized (0.06 g). HPLC t_{R}=2.46 min; LCMS m/z=328, t_{R}=2.18 min (MH⁺); ¹H NMR (300 MHz, CDCl₃): δ 8.04 (dd 1H), 7.77 (dd, 1H), 7.46 (m, 1H), 7.27 (m, 1H), 5.34 (brs., 2H), 4.61 (s, 2H), 4.15 (brs., 1H), 3.10 (m, 2H), 2.90 (s, 3H), 1.65-1.71 (m, 2H), 1.20 (s, 6H), 0.98 (t, 3H); ¹³C NMR (75 MHz, CDCl₃): δ 157.5, 150.8, 144.9, 132.9, 127.5, 126.8, 124.9, 121.9, 119.7, 115.7, 72.4, 58.1, 55.7, 40.6, 27.6, 20.5, 11.5; ¹H NMR (300 MHz, DMSO): δ 8.30 (d, 1H), 7.53 (dd, 1H), 7.32 (m, 1H), 7.14 (m, 1H), 6.26 (brs., 2H), 4.57 (s, 1H), 4.44 (brs., 2H), 3.00 (t, 2H), 2.80 (s, 3H), 1.61 (m, 2H), 1.17 (brs., 6H), 0.92 (t, 3H); ¹³C NMR (75 MHz, DMSO): δ 158.1, 151.1, 144.5, 132.7, 125.9, 125.6, 124.5, 122.0, 119.9, 115.9, 70.9, 57.3, 54.6, 27.8 (very broad), 20.0, 11.4 (one carbon hides in DMSO peak).

### Activity Measurement

### Compound Stimulation and Multi-cytokine Measurement

Human PBMC (hPBMC) (at 1 million cells/ml) or mouse spleen cells (at 5 million cells/ml) or human monocytic THP-1 cells (at 1 million cells/ml) are mixed with tested compounds such as imidazoquinolines at titrated compound concentrations in the complete RPMI medium. After the cell cultures are incubated for 24 hours at 37° C., 5% CO₂, the culture supernatant is collected and assayed for the secreted cytokines in the presence of the compounds. Human or mouse Beadlyte multi-cytokine flex kits (Upstate, Lake Placid, N.Y.) are used to measure the amount of the following cytokines: TNF-α, IL-6, IL-1β, IL-8 and IL-12p40 according to the manufacturers instructions.

### TLR Signaling

HEK293 cells (ATCC, CRL-1573) are seeded in a T75 flask at 3×10⁶ in 20 ml of DMEM supplemented with 0.1 mM nonessential amino acid, 1 mM sodium pyruvate, 2 mM L-glutamine, penicillin-streptomycin, and 10% FCS. After overnight culturing, the cells are transfected with 1) pNFkB-TA-luciferase reporter (0.4 ug) (BD clontech, Palo Alto, Calif.), and with 2) with pGL4.74 (0.01 ug) that carries a TK promoter, not responsive to NF-kB stimulation, and carries a Renilla luciferase gene, used as an internal control (Promega, WI), and 3), separately with a following TLR construct (10 ug): human TLR (hTLR)₇, hTLR8, mouse TLR7 (mTLR7) puno constructs (Invivogene, CA), using Fugene 6 transfection reagent (Roche). The transfected cells after 24 hours transfection are collected and seeded in a 96-well and flat-bottom plate (1×10⁴ cell/well) plate, and stimulated with the test compounds at the following concentrations: 30, 10, 3, 1, 0.3, 0.1, 0.03 uM. After overnight compound stimulation, the cells are assayed for expression of fly and renilla luciferases using Dual-Luciferase Reporter Assay System (Promega, WI). NF-kb activation is directly proportional to relative fly luciferase units, which is measured against the internal control renilla luciferase units.

The contents of each of the patents, patent applications and journal articles cited above are hereby incorporated by reference herein and for all purposes as if fully set forth in their entireties.

1. A method of synthesizing a compound of Formula I: 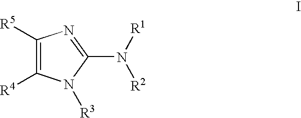
comprising:
reacting a compound of Formula IA: 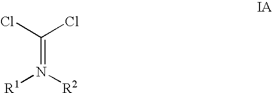
with a compound of Formula IB: 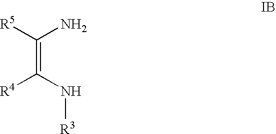
wherein:
R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;
or R¹ and R² taken together form a heterocyclyl or substituted heterocyclyl group;
R³ is selected from the group consisting of H, alkyl, substituted alkyl, hydroxy, alkoxy, substituted alkoxy, amino, substituted amino, acyl, and substituted carbonyl;
R⁴ and R⁵ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;
or R⁴ and R⁵ taken together form a heteroaryl, substituted heteroaryl, cycloalkyl, substituted cycloalkyl, heterocyclyl, or substituted heterocyclyl group.

2. The method of claim 1, wherein the compound of Formula IA further comprises a negatively charged counter ion selected from the group consisting of Cl^{⊖}; F^{⊖}; Br^{⊖}; CF₃SO₃^{⊖}; PCl₆^{⊖}; PF₆^{⊖}; FeCl₄^{⊖}; Cl₃^{⊖}; PO₂Cl₂^{⊖}; ClHCl^{⊖}; Cl(SO₃)₂^{⊖}; ClSO₃^{⊖}; CH₃OSO₃^{⊖}; BF₄^{⊖}; NO₃^{⊖}; SbCl₆^{⊖}; C₂H₅OSO₃^{⊖}; HSO₄^{⊖}; H₂PO₄^{⊖}; CH₃COO^{⊖}; CH₃SO₃^{⊖}; and NO₂.

3. (canceled)

4. The method of claim 1, wherein said step of reacting said compound of Formula IA with said compound of Formula IB is performed in a reaction medium comprising an organic aprotic solvent and a base.

5-8. (canceled)

9. The method of claim 1, wherein R¹ and R² are each independently alkyl or substituted alkyl.

10. The method of claim 1, wherein R³ is alkyl or substituted alkyl.

11. (canceled)

12. The method of claim 1, wherein R⁴ and R⁵ taken together form a heteroaryl or substituted heteroaryl group.

13. The method of claim 12, wherein R⁴ and R⁵ taken together form a quinolinyl or substituted quinolinyl group or a pyridyl or substituted pyridyl group.

14-16. (canceled)

17. The method of claim 12, wherein R⁴ and R⁵ taken together form a heteroaryl group substituted with a halogen; said method further comprising the step of displacing said halogen with an amino or substituted amino group, to form a compound wherein R⁴ and R⁵ taken together form a heteroaryl group substituted with an amino or substituted amino group.

18. A method of synthesizing a compound of Formula II: 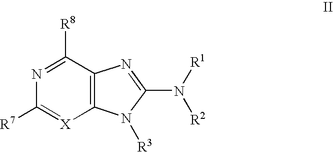
said method comprising the step of:
reacting a compound of Formula IA: 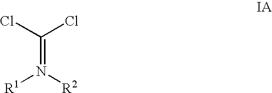
with a compound of Formula IIB: 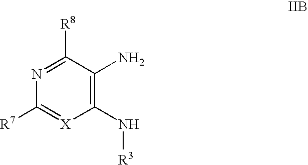
wherein,
X is N or CR⁶;
R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;
or R¹ and R² taken together form a heterocyclyl or substituted heterocyclyl group;
R³ is selected from the group consisting of H, alkyl, hydroxy, alkoxy, substituted alkoxy, substituted alkyl, amino, substituted amino, acyl, and substituted carbonyl;
R⁶ and R⁷ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;
or R⁶ and R⁷ taken together form an aryl, substituted aryl, heteroaryl, substituted heteroaryl, cycloalkyl, substituted cycloalkyl, heterocyclyl, or substituted heterocyclyl group; and
R⁸ is selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

19-25. (canceled)

26. The method of claim 18, wherein R¹ and R² are each independently alkyl or substituted alkyl.

27. The method of claim 18, wherein R³ is alkyl or substituted alkyl.

28. (canceled)

29. The method of claim 18, wherein X is CR⁶.

30. The method of claim 29, wherein R⁶ and R⁷ taken together form a phenyl or substituted phenyl group or a pyridyl or substituted pyridyl group.

31-32. (canceled)

33. The method of claim 18, wherein R⁸ is a halogen, amino, or substituted amino group.

34-35. (canceled)

36. The method of claim 18, wherein R⁸ is a halogen, said method further comprising the step of displacing said halogen with an amino or substituted amino group, to form a compound wherein R⁸ is an amino or substituted amino group.

37. A synthetic method comprising the steps of:
reacting a compound of Formula IA: 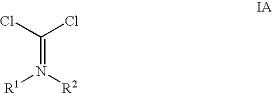
with a compound of Formula IIB: 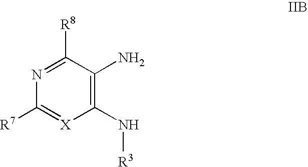
to form a compound of Formula II: 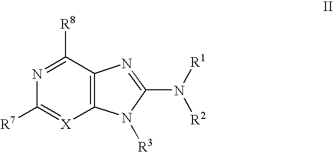
and reacting said compound of Formula II with mCPBA or H₂O₂;
to form a compound of Formula X: 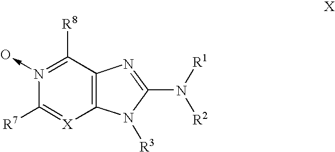
wherein:
X is N or CR⁶;
R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;
or R¹ and R² taken together form a heterocyclyl or substituted heterocyclyl group;
R³ is selected from the group consisting of H, alkyl, hydroxy, alkoxy, substituted alkoxy, substituted alkyl, amino, substituted amino, acyl, and substituted carbonyl;
R⁶ and R⁷ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;
or R⁶ and R⁷ taken together form an aryl, substituted aryl, heteroaryl, substituted heteroaryl, cycloalkyl, substituted cycloalkyl, heterocyclyl, or substituted heterocyclyl group; and
R⁸ is hydrogen.

38. The method of claim 37 further comprising
reacting the compound of Formula X where R⁸ is hydrogen with a halogenating agent, to form a further compound of Formula II wherein R⁸ is a halogen.

39. The method of claim 38, wherein said halogenating agent is POCl₃.

40-48. (canceled)

49. A method of synthesizing a compound of Formula III: 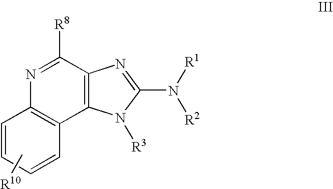
comprising:
reacting a compound of Formula IA: 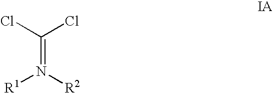
with a compound of Formula IIIB: 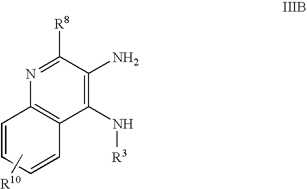
wherein:
R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;
or R¹ and R² taken together form a heterocyclyl or substituted heterocyclyl group;
R³ is selected from the group consisting of H, alkyl, hydroxy, substituted alkyl, alkoxy, substituted alkoxy, amino, substituted amino, acyl, and substituted carbonyl;
R⁸ and R¹⁰ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

50-51. (canceled)

52. The method of claim 49, wherein said step of reacting said compound of Formula IA with said compound of Formula IIIB is performed in a reaction medium comprising an organic aprotic solvent and a base.

53-56. (canceled)

57. The method of claim 49, wherein R¹ and R² are each independently alkyl or substituted alkyl.

58. The method of claim 49, wherein R³ is alkyl or substituted alkyl.

59-61. (canceled)

62. The method of claim 49, wherein R⁸ is a —N(PMB)₂ group, said method further comprising removing said PMB groups from said R⁸, to form an amino group at said R⁸.

63-64. (canceled)

65. The method of claim 49, further comprising
reacting said compound of Formula III with mCPBA or H₂O₂ to form a compound of Formula XI: 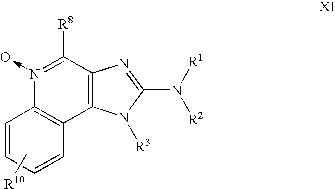
wherein:
R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;
or R¹ and R² taken together form a heterocyclyl or substituted heterocyclyl group;
R³ is selected from the group consisting of H, alkyl, hydroxy, substituted alkyl, alkoxy, substituted alkoxy, amino, substituted amino, acyl, and substituted carbonyl;
R¹⁰ is selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio; and
R⁸ is hydrogen.

66. A synthetic method comprising the steps of:
performing the steps of the method of claim 65; and
reacting the compound of Formula XI where R⁸ is hydrogen with a halogenating agent, to form a further compound of Formula III wherein R⁸ is a halogen.

67. The method of claim 66 further comprising
displacing said halogen R⁸ with an amino group, to form a further compound of Formula III wherein R⁸ is an amino group.

68-72. (canceled)

73. The method of claim 49, wherein a compound of Formula IIIE: 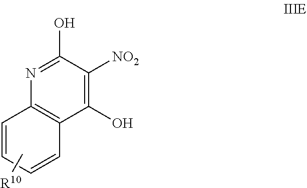
is prepared by a method comprising the step of:
reacting a compound of Formula IIIF: 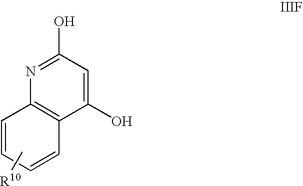
with a nitrosylating agent.

74. The method of claim 73, wherein said nitrosylating agent is HNO₃.

75-77. (canceled)

78. A method of synthesizing a compound of Formula II: 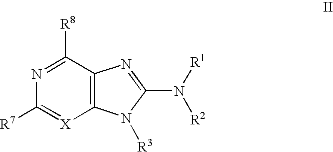
comprising:
reacting a compound of Formula ID: 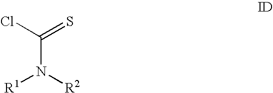
with a compound of Formula IIB: 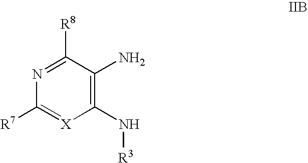
wherein,
X is N or CR⁶;
R¹ and R² are each independently selected from the group consisting of hydrogen, alkyl, substituted alkyl, aryl, substituted aryl, heteroaryl, substituted heteroaryl, heterocyclyl, substituted heterocyclyl, cycloalkyl, and substituted cycloalkyl;
or R¹ and R² taken together form a heterocyclyl or substituted heterocyclyl group;
R³ is selected from the group consisting of H, alkyl, hydroxy, alkoxy, substituted alkoxy, substituted alkyl, amino, substituted amino, acyl, and substituted carbonyl;
R⁶ and R⁷ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio;
or R⁶ and R⁷ taken together form an aryl, substituted aryl, heteroaryl, substituted heteroaryl, cycloalkyl, substituted cycloalkyl, heterocyclyl, or substituted heterocyclyl group; and
R⁸ is selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

79. The method of claim 78, wherein said step of reacting said compound of Formula ID with said compound of Formula IIB is performed in a reaction medium comprising an organic aprotic solvent and a base.

80-82. (canceled)

83. The method of claim 79, wherein said reaction medium further comprises Hg(OAc)₂ and/or said solvent comprises CH₂Cl₂.

84-85. (canceled)

86. The method of claim 78, wherein R¹ is methyl R² is propyl.

87-89. (canceled)

90. The method of claim 78, wherein R⁶ and R⁷ taken together form a phenyl or substituted phenyl group or a pyridyl or substituted pyridyl group.

91-92. (canceled)

93. The method of claim 78, wherein R⁸ is a substituted amino group.

94. The method of claim 93, wherein R⁸ is a —N(PMB)₂ group, further comprising removing said PMB groups from said R⁸ to form an amino group at said R⁸.

95-109. (canceled)

110. A method of synthesizing a compound of Formula III: 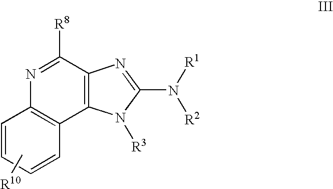
comprising:
reacting a compound of Formula ID: 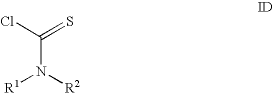
with a compound of Formula IIIB: 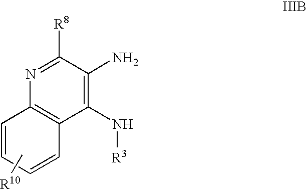
wherein:
R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;
or R¹ and R² taken together form a heterocyclyl or substituted heterocyclyl group;
R³ is selected from the group consisting of H, alkyl, hydroxy, substituted alkyl, alkoxy, substituted alkoxy, amino, substituted amino, carbonyl, and substituted carbonyl;
R⁸ and R¹⁰ are each independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio.

111. The method of claim 110, wherein said step of reacting said compound of Formula ID with said compound of Formula IIIB is performed in a reaction medium comprising an organic aprotic solvent and a base.

112-114. (canceled)

115. The method of claim 111, wherein said reaction medium further comprises Hg(OAc)₂.

116. (canceled)

117. The method of claim 110, wherein R¹ and R² are both independently alkyl or substituted alkyl.

118-120. (canceled)

121. The method of claim 110, wherein R¹⁰ is hydrogen.

122. The method of claim 110, wherein R⁸ is a substituted amino group.

123. The method of claim 122, wherein R⁸ is a —N(PMB)₂ group.

124-125. (canceled)

126. The method of claim 110, further comprising
reacting said compound of Formula III with mCPBA or H₂O₂ to form a compound of Formula XI: 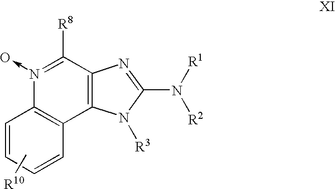
wherein:
R¹ and R² are each independently selected from the group consisting of alkyl, substituted alkyl, aryl, substituted aryl, heteroaryl, substituted heteroaryl, heterocyclyl, substituted heterocyclyl, cycloalkyl, and substituted cycloalkyl;
or R¹ and R² taken together form a heterocyclyl or substituted heterocyclyl group;
R³ is selected from the group consisting of H, alkyl, hydroxy, substituted alkyl, alkoxy, substituted alkoxy, amino, substituted amino, carbonyl, and substituted carbonyl;
R¹⁰ is selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, and substituted alkylthio; and
R⁸ is hydrogen.

127. The method of claim 126, further comprising
reacting said compound of Formula XI with a halogenating agent, to form a further compound of Formula III wherein R⁸ is a halogen.

128. The method of claim 127, wherein said halogenating agent is POCl₃.

129-130. (canceled)

131. The method of claim 110, further comprising the step of reacting the compound of Formula IIID with HN(PMB)₂, to form a compound wherein R⁸ is —N(PMB)₂.

132-134. (canceled)

135. The method of claim 110, wherein a compound of Formula IIIE: 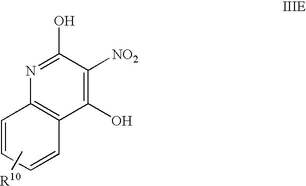
is prepared by a method comprising the step of:
reacting a compound of Formula IIIF: 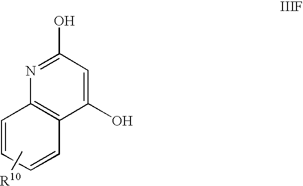
with a nitrosylating agent.

136. The method of claim 135, wherein said nitrosylating agent is HNO₃.

137-140. (canceled)
